SEQUENCE LISTING (6 sequences)

SEQ ID NO: 1 - protein, 5 aa, Artificial Sequence
  sequence motif
RSASR

SEQ ID NO: 2 - protein, 5 aa, Artificial Sequence
  sequence motif
RTASR

SEQ ID NO: 3 - protein, 5 aa, Artificial Sequence
  sequence motif
RSRAR

SEQ ID NO: 4 - protein, 5 aa, Artificial Sequence
  sequence motif
RTRAR

SEQ ID NO: 5 - protein, 5 aa, Artificial Sequence
  sequence motif
ATATR

SEQ ID NO: 6 - protein, 6 aa, Artificial Sequence
  sequence motif
RSAASR

(19) United States
(12) Patent Application Publication
(10) Pub. No.: US 2003/0119770 A1
(43) Pub. Date: Jun. 26, 2003

(54) Intercellular delivery of a herpes simplex virus VP22 fusion protein from cells infected with lentiviral vectors

(76) Inventors: Zhennan Lai, N. Potomac, MD (US); Jakob Reiser, New Orleans, LA (US); Roscoe O. Brady, Rockville, MD (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/212,634

(22) Filed: Aug. 2, 2002

Related U.S. Application Data
(60) Provisional application No. 60/310,012, filed on Aug. 2, 2001.

(51) Int. Cl. A61K 48/00; C12N 7/00; C12N 15/867

(52) U.S. Cl. 514/44; 424/93.2; 435/456; 435/320.1; 435/235.1

(57) ABSTRACT

The present invention is related to use of recombinant lentiviral vectors containing a therapeutic gene of interest fused in-frame with an intercellular trafficking gene for the global delivery of therapeutic proteins in nondividing cells.

### CROSS-REFERENCE TO RELATED APPLICATIONS

This application claims the benefit of priority of U.S. provisional patent application No. 60/310,012, filed Aug. 2, 2001, which is hereby expressly incorporated by reference in its entirety.

### FIELD OF THE INVENTION

The present invention is related to use of recombinant lentiviral vectors containing a therapeutic gene of interest fused in-frame with an intercellular trafficking gene for the global delivery of therapeutic proteins in nondividing cells.

### BACKGROUND OF THE INVENTION

A number of obstacles currently limit the effectiveness of gene therapy. One of the most formidable is the delivery of desired genes or proteins to a sufficient number of target cells to elicit a therapeutic response. Recently, a series of virus-encoded and other regulatory proteins were found to possess the ability to cross biological membranes. For example, peptides derived from the Drosophila Antennapedia homeodomain are internalized by cells in culture (Derossi, D. et al. 1994 J Biol Chem 269:10444-10450; Derossi, D. et al. 1996 J Biol Chem 271:18188-18193) and conveyed to cell nuclei where they can directly and specifically interfere with transcription (Derossi, D. et al. 1996 J Biol Chem 271:18188-18193; Le Roux, I. et al 1995 FEBS Lett 368:311-314). The HIV-1 Tat protein was reported to enhance intercellular trafficking in vitro (Frankel, A. D. & Pabo, C. O. 1988 Cell 55:1189-1193; Green, M. & Loewenstein, P. M. 1988 Cell 55:1179-1188). The Tat protein is composed of 86 amino acids and contains a highly basic region and a cysteine-rich region (Frankel, A. D. & Pabo, C. O. 1988 Cell 55:1189-1193). It was found that Tat-derived peptides as short as 11 amino acids are sufficient for transduction of proteins (Fawell, S. et al. 1994 PNAS USA 91:664-668; Nagahara, H. et al. 1998 Nat Med 4: 1449-1452). However, the exact mechanism by which the 11-amino acid transduction domain crosses lipid bilayers is poorly understood. Schwarze et al. (Schwarze, R. S. et al. 1999 Science 385:1569-1572) have recently generated a Tat-galactosidase fusion protein that was delivered efficiently into brain tissue and skeletal muscle in vivo. These findings suggest that protein therapies may be successfully developed provided that problems caused by immune response and toxicity that might be associated with long-term expression of novel proteins in vivo can be solved.

The herpes simplex virus type 1 tegument protein VP22 was also reported to exhibit a unique property of effecting intercellular spread. VP22-directed delivery of proteins could be achieved either by transfection of genes encoding VP22 or by exogenous application of a protein extract containing VP22 (Elliott, G. & O'Hare, P. 1997 Cell 88:223-233). VP22 is a basic, 38-kDa phosphorylated protein (Knopf, K. W. & Kaemer, H. C. 1980 J Gen Virol 46:405-414) encoded by the viral UL49 gene (Elliott, G. D. & Meredith, D. M. 1992 J Gen Virol 73:723-726). The transport of VP22 occurs via a mechanism potentially involving actin microfilaments. VP22 is exported from the cytoplasm of expressing cells and imported into neighboring cells where it accumulates in the nucleus (Elliott, G. & O'Hare, P. 1997 Cell 88:223-233). These properties aroused interest in VP22 as a delivery vehicle for therapeutic proteins (Dilber, M. S. et al. 1999 Gene Ther 6:12-21). Recent studies suggest that VP22 is distributed to at least three distinct subcellular locations, which were defined as nuclear, diffuse, and cytoplasmic (Pomeranz, L. & Blaho, J. 1999 J Virol 73:6769-6781). All of the data obtained thus far were based on studies with transfected cells in culture. The delivery of a recombinant fusion protein by a lentiviral vector into the brain in vivo has not been reported.

### SUMMARY OF THE INVENTION

The present invention is related to use of recombinant lentiviral vectors containing a therapeutic gene of interest fused in-frame with an intercellular trafficking gene for the global delivery of therapeutic proteins in nondividing cells.

### BRIEF DESCRIPTION OF THE DRAWINGS

FIG. 1 shows HIV-1-based gene transfer systems. (A) Helper (packaging) construct. The triangle symbolizes a deletion affecting the packaging signal between the 5′ splice donor site and the beginning of the gag sequence. The poly(A) site was derived from the bovine growth hormone gene. (B) Transducing vector constructs. The HIV-EGFP/HSA (i) and HIV-VP22 EGFP/HSA (ii) constructs are shown. Boxes interrupted by jagged lines contain partial deletions. CMV, Human CMV-IE promoter. (C) Env expression construct encoding vesicular stomatitis virus G glycoprotein (VSV-G). VSV-G expression is driven by the HIV-1 LTR. The poly(A) site was derived from the simian virus 40 late region. EGFP, enhanced green fluorescent protein; HSA, heat-stable antigen.

FIG. 2 shows HIV-1-based gene transfer vectors. Boxes interrupted by jagged lines contain partial deletions. Abbreviations: P, heterologous transcription promoter; SD, splice donor site; SA, splice acceptor site.

FIG. 3 shows an EGFP expression cassette consisting of EGFP sequences and the CMV IE promoter which was inserted within the viral env-coding region. HSA sequences were inserted at the 5′ end of nef

FIG. 4 shows an enhanced green fluorescent protein (EGFP) expression cassette consisting of EGFP sequences and the CMV IE promoter which was inserted within the viral gag-pol coding region. A second expression cassette consisting of neo sequences driven by the SV40 early promoter was placed within the env-coding region. HSA sequences were inserted at the 5′ end of nef.

FIG. 5 shows vector construct containing EGFP and HSA reporter genes linked by the ECMV IRES.

FIG. 6 shows vector constructs containing the CMV IE or CEF promoter and an ECMV or Gtx IRES element.

FIG. 7 is a diagrammatic illustration of the recombinant lentiviral vector. (A) Vector construct contains reporter gene encoding EGFP driven by a CMV promoter. (B) A NSE promoter is inserted into the lentiviral vector to replace the CMV promoter.

FIG. 8 shows the in vivo distribution of EGFP-positive cells in the central nervous system. The numbers of EGFP-positive cells in striatum (A) and hippocampus (B) were counted by laser scanning under the confocal microscopy and were analyzed three-dimensionally with a computer program. The statistical evaluation for the data was performed using a Student's unpaired t-test, the values are means±S.D. (n=5; *P<0.05)

### DETAILED DESCRIPTION OF THE PREFERRED EMBODIMENT

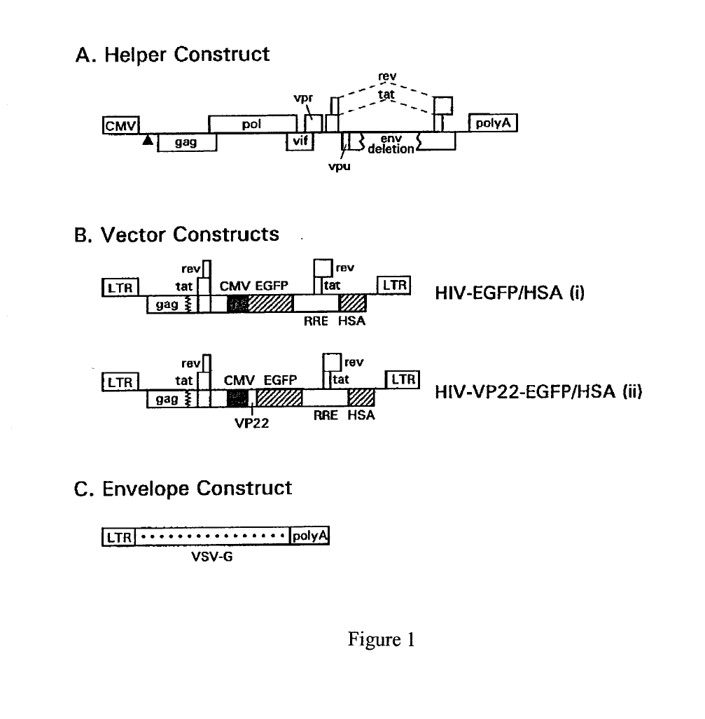

Effective gene therapy depends on the efficient transfer of therapeutic genes and their protein products to target cells. Lentiviral vectors appear promising for virus-mediated gene delivery and long-term expression in nondividing cells. The herpes simplex virus type 1 tegument protein VP22 has recently been shown to mediate intercellular transport of proteins, raising the possibility that it may be helpful in a setting where the global delivery of therapeutic proteins is desired. Referring to FIG. 1, to investigate the effectiveness of lentiviral vectors to deliver genes encoding proteins fused to VP22, and to test whether the system is sufficiently potent to allow protein delivery from transduced cells in vitro and in vivo, fusion constructs of VP22 and enhanced green fluorescent protein (EGFP) were prepared and delivered into target cells by using HIV-1-based lentiviral vectors. To follow the spread of VP22-EGFP to other cells, transduced COS-7 cells were coplated with a number of different cell types, including brain choroid plexus cells, human endothelial cells, H9 cells, and HeLa cells. We found that VP22-EGFP fusion proteins were transported from transduced cells to recipient cells and that such fusion proteins accumulated in the nucleus and in the cytoplasm of such cells. To determine the ability to deliver fusion proteins in vivo, we injected transduced H9 cells as well as the viral vector directly into the brain of mice. We observed that VP22-EGFP fusion proteins were transported effectively from lentivirus transduced cells in vivo. We also found that the VP22-EGFP fusion protein encoded by the lentivirus is transported between cells. Our data indicated that such fusion proteins are present in the nucleus and in the cytoplasm of neighboring cells. Injection of the viral vector directly into the brain of mice resulted in delivery of VP22-EGFP fusion protein to many neighboring cells of mouse brain. Therefore, lentiviral vectors provide a potent biological system for delivering genes encoding therapeutic proteins fused to VP22.

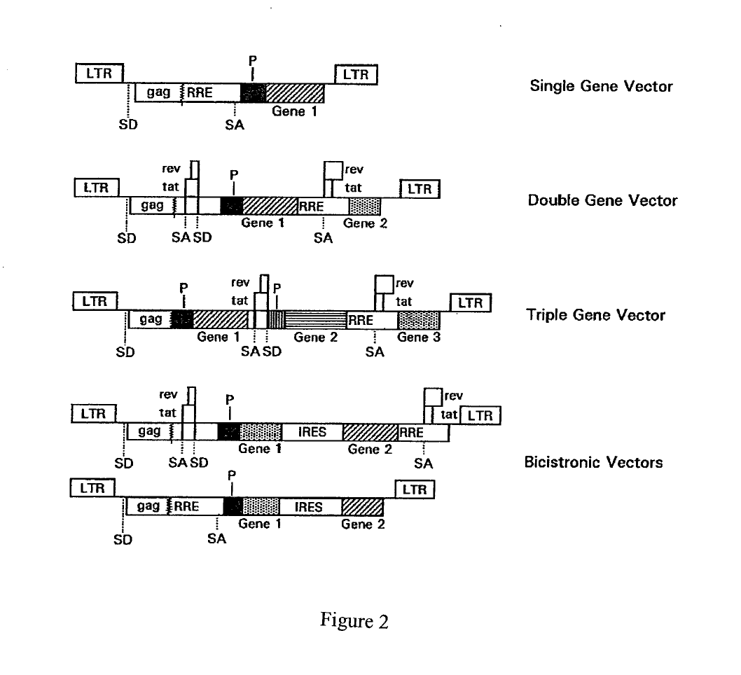

Previously we described safe and efficient three-component human immunodeficiency virus type 1 (HIV-1)-based gene transfer systems for delivery of genes into nondividing cells (Mochizuki, H. et al. 1998 J Virol 72:8873-8883). Referring to FIG. 2, 3, 4, 5, and 6, to apply such vectors in anti-HIV gene therapy strategies and to express multiple proteins in single target cells, we have engineered HIV-1 vectors for the concurrent expression of multiple transgenes. Single-gene vectors, bicistronic vectors, and multigene vectors expressing up to three exogenous genes under the control of two or three different transcriptional units, placed within the viral gag-pol coding region and/or the viral nef and env genes, were designed. These versatile vectors can be used in a wide variety of gene therapy applications.

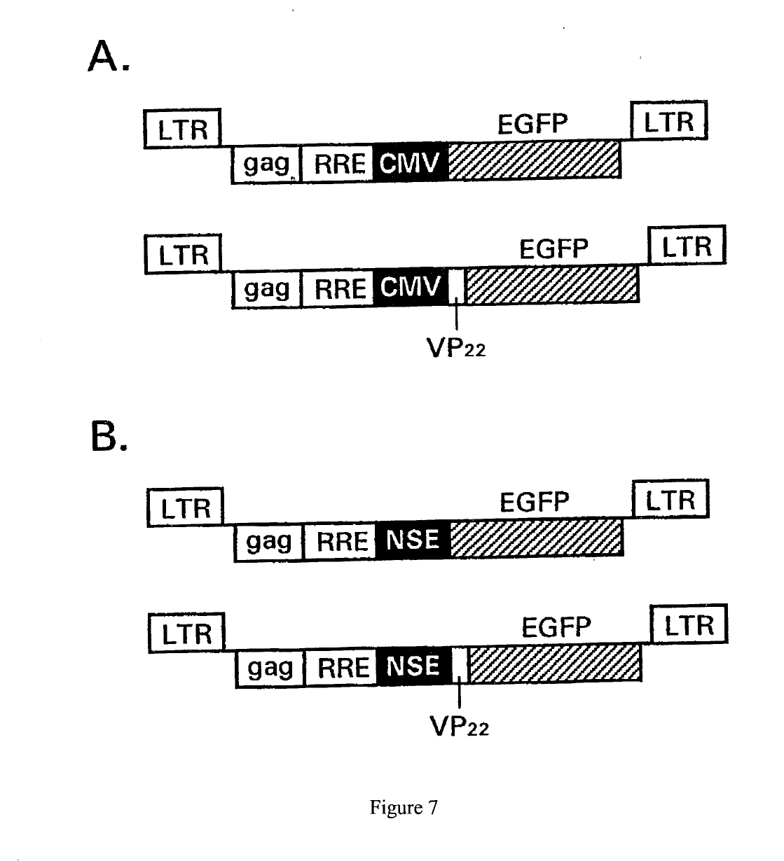

Gene transfer vectors derived from human immunodeficiency virus (HIV-1) efficiently transduce nondividing cells and may provide for the delivery of their gene products to discrete regions of the brain. Referring to FIG. 7, we investigated whether stable gene transduction can be achieved in cells of the central nervous system (CNS) in vivo by a potent lentivirus vector. The herpes simplex virus type 1 protein VP22 has been known to facilitate intercellular protein transport and thereby provides an opportunity to increase the effectiveness of therapeutic genes by enhancing the delivery of their protein products. We developed a lentiviral vector construct expressing enhanced green fluorescent protein (EGFP) fused at its N-terminus to the herpes simplex virus VP22. In order to determine expression of the fusion protein in specific cells such as neurons in the CNS, a neuron specific promoter was also placed into the lentiviral vector construct. The viral vectors were injected directly into the striatum and hippocampus of mouse brains. We found that the lentivirus vector efficiently and stably transduced nondividing cells in CNS with transgene expression for over 3 months. We also found that the delivery of VP22-EGFP fusion protein encoded by the lentivirus was effectively transported between neuronal cells via axons in vivo. Doubly labeled experiments revealed that our lentiviral vector is capable of delivering gene products to neurons and astrocytes in CNS. Our data also demonstrate that up to 90% of the CNS cells transduced by our lentiviral vector under the control of neuronal promoters are neurons.

Vectors and Methods of Use for Nucleic Acid Delivery to Non-Dividing Cells

The present invention provides a recombinant lentivirus capable of infecting non-dividing cells. The virus is useful for the in vivo and ex vivo transfer and expression of genes nucleic acid sequences (e.g., in non-dividing cells).

Lentiviruses are RNA viruses wherein the viral genome is RNA. When a host cell is infected with a lentivirus, the genomic RNA is reverse transcribed into a DNA intermediate which is integrated very efficiently into the chromosomal DNA of infected cells. This integrated DNA intermediate is referred to as a provirus. Transcription of the provirus and assembly into infectious virus occurs in the presence of an appropriate helper virus or in a cell line containing appropriate sequences enabling encapsidation without coincident production of a contaminating helper virus. As described below, a helper virus is not required for the production of the recombinant lentivirus of the present invention, since the sequences for encapsidation are provided by co-transfection with appropriate vectors.

The lentiviral genome and the proviral DNA have three genes: the gag, the pol, and the env, which are flanked by two long terminal repeat (LTR) sequences. The gag gene encodes the internal structural (matrix, capsid, and nucleocapsid) proteins; the pol gene encodes the RNA-directed DNA polymerase (reverse transcriptase) and the env gene encodes viral envelope glycoproteins. The 5′ and 3′ LTRs serve to promote transcription and polyadenylation of the virion RNAs. The LTR contains all other cis-acting sequences necessary for viral replication. Lentiviruses have additional genes including vit, vpr, tat, rev, vpu, nef, and vpx (in HIV-1, HIV-2 and/or SIV).

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of lentiviral RNA into infectious virions) are missing from the viral genome, the result is a cis defect which prevents encapsidation of genomic RNA. However, the resulting mutant is still capable of directing the synthesis of all virion proteins.

In a first embodiment, the invention provides a recombinant lentivirus capable of infecting a non-dividing cell. The recombinant lentivirus comprises a nucleic acid sequence containing a lentiviral packaging signal flanked by lentiviral cis-acting nucleic acid sequences necessary for reverse transcription and integration, a heterologous nucleic acid sequence operably linked to a regulatory nucleic acid sequence, and a nucleic acid sequence encoding an intercellular trafficking signal, where the nucleic acid sequence encoding the intercellular trafficking signal is fused in-frame with the heterologous nucleic acid sequence, where the lentivirus does not contain either a complete gag, pol, or env gene. It should be understood that the recombinant lentivirus of the invention is capable of infecting dividing cells as well as non-dividing cells.

The recombinant lentivirus of the invention is therefore genetically modified in such a way that some of the structural, infectious genes of the native virus have been removed and replaced instead with a nucleic acid sequence to be delivered to a target non-dividing cell. After infection of a cell by the virus, the virus releases its nucleic acid into the cell and the lentivirus genetic material can integrate into the host cell genome. The transferred lentivirus genetic material is then transcribed and translated into proteins within the host cell.

The invention provides a method of producing a recombinant lentivirus capable of infecting a non-dividing cell comprising transfecting a suitable host cell with the following: a first vector providing a nucleic acid encoding a lentiviral gag and a lentiviral pol, where the gag and pol nucleic acid sequences are operably linked to a heterologous regulatory nucleic acid sequence and where the first vector is defective for nucleic acid sequence encoding functional env protein and devoid of lentiviral sequences both upstream and downstream from a splice donor site to a gag initiation site of a lentiviral genome; a second vector providing a nucleic acid encoding a non-lentiviral env protein; and a third vector providing a nucleic acid sequence containing a lentiviral packaging signal flanked by lentiviral cis-acting nucleic acid sequences for reverse transcription and integration, and providing a cloning site for introduction of a heterologous nucleic acid sequence operably linked to a regulatory nucleic acid sequence and a nucleic acid sequence encoding an intercellular trafficking signal, where the nucleic acid sequence encoding the intercellular trafficking signal is fused in-frame with the heterologous nucleic acid sequence, where the third vector does not contain either a complete gag, pol, or env gene, and recovering the recombinant lentivirus. An illustration of the individual vectors used in the method of the invention is shown in FIG. 1.

The method of the invention includes the combination of a minimum of three vectors in order to produce a recombinant virion or recombinant lentivirus.

A first vector provides a nucleic acid encoding a lentiviral gag and a lentiviral pol. See FIG. 1.

A second vector provides a nucleic acid encoding a non-lentiviral env protein. See FIG. 1. The env gene can be derived from any virus excluding lentiviruses. For public policy reasons, since a lentivirus is an HIV, the env will be derived from a virus other than HIV. The env may be amphotropic envelope protein which allows transduction of cells of human and other species, or may be ecotropic envelope protein, which is able to transduce only mouse and rat cells. Further, it may be desirable to target the recombinant virus by linkage of the envelope protein with an antibody or a particular ligand for targeting to a receptor of a particular cell-type. By inserting a sequence (including regulatory region) of interest into the viral vector, along with another gene which encodes the ligand for a receptor on a specific target cell, for example, the vector is now target specific. Lentiviral vectors can be made target specific by inserting, for example, a protein. Targeting is often accomplished by using an antibody to target the lentiviral vector. Those of skill in the art will know of, or can readily ascertain without undue experimentation, specific methods to achieve delivery of a lentiviral vector to a specific target.

Examples of retroviral-derived env genes include, but are not limited to: Moloney murine leukemia virus (MoMuLV), Harvey murine sarcoma virus (HaMuSV), murine mammary tumor virus (MuMTV), gibbon ape leukemia virus (GaLV), and Rous Sarcoma Virus (RSV). Other env genes such as Vesicular stomatitis virus (VSV) (Protein G) can also be used.

The vector providing the viral env nucleic acid sequence is operably associated with regulatory sequence, e.g., a promoter or enhancer. Preferably, the regulatory sequence is a viral promoter. The regulatory sequence can be any eukaryotic promoter or enhancer, including for example, the Moloney murine leukemia virus promoter-enhancer element, the human cytomegalovirus enhancer, or the vaccinia P7.5 promoter. In some cases, such as the HIV-1 promoter-enhancer element, these promoter-enhancer elements are located within or adjacent to the LTR sequences.

A third vector provides a nucleic acid sequence contains the cis-acting viral sequences necessary for the lentiviral life cycle. Such sequences include the lentiviral psi packaging sequence, reverse transcription signals, integration signals, viral promoter, enhancer, and polyadenylation sequences. The third vector also contains a cloning site for a heterologous nucleic acid sequence to be transferred to a non-dividing cell, and a nucleic acid sequence encoding an intercellular trafficking signal, where the nucleic acid sequence encoding the intercellular trafficking signal is fused in-frame with the heterologous nucleic acid sequence. See FIG. 1.

Since recombinant lentiviruses produced by standard methods in the art are defective, they require assistance in order to produce infectious vector particles. Typically, this assistance is provided, for example, by using a helper cell line that provides the missing viral functions. These plasmids are missing a nucleotide sequence which enables the packaging mechanism to recognize an RNA transcript for encapsidation. Suitable cell lines produce empty virions, since no genome is packaged. If a lentiviral vector is introduced into such cells in which the packaging signal is intact, but the structural genes are replaced by other genes of interest, the vector can be packaged and vector virion produced.

The method of producing the recombinant lentivirus of the invention is different than the standard helper virus/packaging cell line method described above. The three or more individual vectors used to co-transfect a suitable packaging cell line collectively contain all of the required genes for production of a recombinant virus for infection and transfer of nucleic acid to a non-dividing cell. Consequently, there is no need for a helper virus.

The heterologous nucleic acid sequence is operably linked to a regulatory nucleic acid sequence. As used herein, the term “heterologous” nucleic acid sequence refers to a sequence that originates from a foreign species, or, if from the same species, it may be substantially modified from its original form. Alternatively, an unchanged nucleic acid sequence that is not normally expressed in a cell is a heterologous nucleic acid sequence. The term “operably linked” refers to functional linkage between the regulatory sequence and the heterologous nucleic acid sequence. Preferably, the heterologous sequence is linked to a promoter, resulting in a chimeric gene. The heterologous nucleic acid sequence is preferably under control of either the viral LTR promoter-enhancer signals or of an internal promoter, and retained signals within the lentiviral LTR can still bring about efficient integration of the vector into the host cell genome.

The promoter sequence may be homologous or heterologous to the desired gene sequence. A wide range of promoters may be utilized, including viral or mammalian promoters. Cell or tissue specific promoters can be utilized to target expression of gene sequences in specific cell populations. Suitable mammalian and viral promoters for the present invention are available in the art.

Conveniently during the cloning stage, the nucleic acid construct referred to as the transfer vector, having the packaging signal and the heterologous cloning site, also contains a selectable marker gene. Marker genes are utilized to assay for the presence of the vector, and thus, to confirm infection and integration. Typical selection genes encode proteins that confer resistance to antibiotics and other toxic substances, e.g. histidinol, puromycin, hygromycin, neomycin, methotrexate, etc.

By “intercellular trafficking signal” is meant an amino acid sequence that imparts the property to a protein of being able to pass through membranes between cells. Examples of membrane-penetrating proteins include, but are not limited to, several plant and bacterial protein toxins, such as ricin, abrin, modeccin, diphtheria toxin, cholera toxin, anthrax toxin, heat labile toxins, and Pseudomonas aeruginosa exotoxin A. Examples of membrane-penetrating proteins that are not toxins include the TAT protein of human immunodeficiency virus and the protein VP22, the product of the UL49 gene of herpes simplex virus type 1. One line of research involves adapting such molecules from their naturally destructive role into therapeutic compositions. If this can be accomplished, nature may have already provided a valuable starting point for the improvement of molecular therapies.

In this specification, “VP22” denotes: protein VP22 of HSV, e.g., of HSV1, and transport-active fragments and homologues thereof, including transport-active homologues from other herpesviruses including varicella zoster virus VZV, marek's disease virus MDV and bovine herpesvirus BHV.

Among sub-sequences of herpesviral VP22 protein with transport activity, investigators have found that for example transport activity is present in polypeptides corresponding to aminoacids 60-301 and 159-301 of the full HSV1 VP22 sequence (1-301). A polypeptide consisting of aa 175-301 of the VP22 sequence has markedly less transport activity, and is less preferred in connection with the present invention. Accordingly, the present invention relates in one aspect to a sub-sequence of VP22 containing a sequence starting preferably from about aa 159 (or earlier, towards the N-terminal, in the native VP22 sequence), to about aa 301, and having (relative to the full VP22 sequence) at least one deletion of at least part of the VP22 sequence which can extend for example from the N-terminal to the cited starting point, e.g., a deletion of all or part of the sequence of about aa 1-158. (Less preferably, such a deletion can extend further in the C-terminal direction, e.g., to about aa 175.) For example, partial sequences in the range from about aa 60-301 to about aa 159-301 are provided.

VP22 sequences as contemplated herein extend to homologous proteins and fragments based on sequences of VP22 protein homologues from other herpesviruses, e.g., the invention provides corresponding derivatives and uses of the known VP22-homologue sequences from VZV (e.g., all or homologous parts of the sequence from aa 1-302), from MDV (e.g., all or homologous parts of the sequence from aa 1-249) and from BHV (e.g., all or homologous parts of the sequence from aa 1-258). The sequences of the corresponding proteins from HSV2, VZV, BHV and MDV are available in public protein/nucleic acid sequence databases. Thus, for example, within the EMBL/Genbank database, a VP22 sequence from HSV2 is available as gene item UL49 under accession no. Z86099 containing the complete genome of HSV2 strain HG52; the complete genome of VZV including the homologous gene/protein is available under accession numbers X04370, M14891, M16612; the corresponding protein sequence from BHV is available as “bovine herpesvirus 1 virion tegument protein” under accession number U21137; and the corresponding sequence from MDV is available as gene item UL49 under accession number L10283 for “gallid herpesvirus type 1 homologous sequence genes”. In these proteins, especially those from HSV2 and VZV, corresponding deletions can be made, e.g., of sequences homologous to aa 1-159 of VP22 from HSV1. Homologies between these sequences are readily accessible by the use of standard algorithms, default parameters, and software.

Furthermore, chimeric VP22 proteins and protein sequences are also useful within the context of the present invention, e.g., a protein sequence from VP22 of HSV1 for part of which a homologous sequence from the corresponding VP22 homologue of another herpesvirus has been substituted. For example, into the sequence of polypeptide 159-301 from VP22 of HSV1, C-terminal sequences can be substituted from VP22 of HSV2 or from the VP22 homologue of BHV.

Investigators have found that deletion of the 34-amino acid C-terminal sequence from VP22 of HSV1 abolishes transport-activity, thus this sequence region contains essential elements for transport activity. According to a further aspect of the invention, there are provided in-frame fusions comprising a nucleic acid sequence encoding the 34-amino acid C-terminal sequence from VP22, or a variant thereof, together with a sequence for a heterologous nucleic acid sequence. In-frame fusions of nucleic acid sequences encoding modified terminal fragments having at least one mutation insertion or deletion relative to the C-terminal 34 amino acid sequence of HSV1 VP22 are also provided.

Investigators have also been found that sequences necessary for transport activity contain one or a plurality of amino acid sequence motifs or their homologues from the C-terminal sequence of VP22 of HSV1 or other herpesviruses, which can be selected from RSASR (SEQ ID NO: 1), RTASR (SEQ ID NO: 2), RSRAR (SEQ ID NO: 3), RTRAR (SEQ ID NO: 4), ATATR (SEQ ID NO 5), and wherein the third or fourth residue A can be duplicated, e.g., as in RSAASR (SEQ ID NO: 6). Corresponding in-frame fusions of nucleic acid sequences encoding these signals are also provided.

The recombinant virus of the invention is capable of transferring a nucleic acid sequence into a non-dividing cell. The term nucleic acid sequence refers to any nucleic acid molecule, preferably DNA. The nucleic acid molecule may be derived from a variety of sources, including DNA, cDNA, synthetic DNA, RNA, or combinations thereof. Such nucleic acid sequences may comprise genomic DNA which may or may not include naturally occurring introns. Moreover, such genomic DNA may be obtained in association with promoter regions, introns, or poly(A) sequences. Genomic DNA may be extracted and purified from suitable cells by means well known in the art. Alternatively, messenger RNA (mRNA) can be isolated from cells and used to produce cDNA by reverse transcription or other means.

The phrase “non-dividing” cell refers to a cell that does not go through mitosis. Non-dividing cells may be blocked at any point in the cell cycle, (e.g., G₀/G₁, G₁/S, G₂/M), as long as the cell is not actively dividing. For ex vivo infection, a dividing cell can be treated to block cell division by standard techniques used by those of skill in the art, including, irradiation, aphidocolin treatment, serum starvation, and contact inhibition. However, it should be understood that ex vivo infection is often performed without blocking the cells since many cells are already arrested (e.g., stem cells). The recombinant lentivirus vector of the invention is capable of infecting any non-dividing cell, regardless of the mechanism used to block cell division or the point in the cell cycle at which the cell is blocked. Examples of pre-existing non-dividing cells in the body include neuronal, muscle, liver, skin, heart, lung, and bone marrow cells, and their derivatives.

The method of the invention provides at least three vectors which provide all of the functions required for packaging of recombinant virions as discussed above. The method also envisions transfection of vectors including viral genes such as vpr, vif, nef, vpx, tat, rev, and vpu. Some or all of these genes can be included, for example, on the packaging construct vector, or, alternatively, they may reside on individual vectors. There is no limitation to the number of vectors which are utilized, as long as they are co-transfected to the packaging cell line in order to produce a single recombinant lentivirus. For example, one could put the env nucleic acid sequence on the same construct as the gag and pol.

The vectors are introduced via transfection or infection into the packaging cell line. The packaging cell line produces viral particles that contain the vector genome. Methods for transfection or infection are well known by those of skill in the art. After co-transfection of the at least three vectors to the packaging cell line, the recombinant virus is recovered from the culture media and titered by standard methods used by those of skill in the art.

In another embodiment, the invention provides a recombinant lentivirus produced by the method of the invention as described above.

The invention also provides a method of nucleic acid transfer to a non-dividing cell to provide expression of a particular nucleic acid sequence. Therefore, in another embodiment, the invention provides a method for introduction and expression of a heterologous nucleic acid sequence in a non-dividing cell comprising infecting the non-dividing cell with the recombinant virus of the invention and expressing the heterologous nucleic acid sequence in the non-dividing cell.

It may be desirable to modulate the expression of a gene regulating molecule in a cell by the introduction of a molecule by the method of the invention. The term “modulate” envisions the suppression of expression of a gene when it is over-expressed, or augmentation of expression when it is under-expressed. Where a cell proliferative disorder is associated with the expression of a gene, nucleic acid sequences that interfere with the gene's expression at the translational level can be used. This approach utilizes, for example, antisense nucleic acid, ribozymes, or triplex agents to block transcription or translation of a specific mRNA, either by masking that mRNA with an antisense nucleic acid or triplex agent, or by cleaving it with a ribozyme.

Antisense nucleic acids are DNA or RNA molecules that are complementary to at least a portion of a specific mRNA molecule (Weintraub, 1990 Scientific American 262:40). In the cell, the antisense nucleic acids hybridize to the corresponding mRNA, forming a double-stranded molecule. The antisense nucleic acids interfere with the translation of the mRNA, since the cell will not translate a mRNA that is double-stranded. Antisense oligomers of about 15 nucleotides are preferred, since they are easily synthesized and are less likely to cause problems than larger molecules when introduced into the target cell. The use of antisense methods to inhibit the in vitro translation of genes is well known in the art (Marcus-Sakura, 1988 Anal Biochem 172:289).

The antisense nucleic acid can be used to block expression of a mutant protein or a dominantly active gene product, such as amyloid precursor protein that accumulates in Alzheimer's disease. Such methods are also useful for the treatment of Huntington's disease, hereditary Parkinsonism, and other diseases. Antisense nucleic acids are also useful for the inhibition of expression of proteins associated with toxicity.

Use of an oligonucleotide to stall transcription is known as the triplex strategy since the oligomer winds around double-helical DNA, forming a three-strand helix. Therefore, these triplex compounds can be designed to recognize a unique site on a chosen gene (Maher, et al. 1991 Antisense Res and Dev 1:227; Helene, C. 1991 Anticancer Drug Design 6:569).

Ribozymes are RNA molecules possessing the ability to specifically cleave other single-stranded RNA in a manner analogous to DNA restriction endonucleases. Through the modification of nucleotide sequences which encode these RNAs, it is possible to engineer molecules that recognize specific nucleotide sequences in an RNA molecule and cleave it (Cech, 1988 J Amer Med Assn 260:3030). A major advantage of this approach is that, because they are sequence-specific, only mRNAs with particular sequences are inactivated.

It may be desirable to transfer a nucleic acid encoding a biological response modifier. Included in this category are immunopotentiating agents including nucleic acids encoding a number of the cytokines classified as “interleukins”. These include, for example, interleukins 1 through 12. Also included in this category, although not necessarily working according to the same mechanisms, are interferons, and in particular gamma interferon (γ-IFN), tumor necrosis factor (TNF) and granulocyte-macrophage-colony stimulating factor (GM-CSF). It may be desirable to deliver such nucleic acids to bone marrow cells or macrophages to treat enzymatic deficiencies or immune defects. Nucleic acids encoding growth factors, toxic peptides, ligands, receptors, or other physiologically important proteins can also be introduced into specific non-dividing cells.

The recombinant lentivirus of the invention can be used to treat an HIV infected cell (e.g., T cell or macrophage) with an anti-HIV molecule. In addition, respiratory epithelium, for example, can be infected with a recombinant lentivirus of the invention having a gene for cystic fibrosis transmembrane conductance regulator (CFTR) for treatment of cystic fibrosis.

The method of the invention may also be useful for neuronal or glial cell transplantation, or “grafting”, which involves transplantation of cells infected with the recombinant lentivirus of the invention ex vivo, or infection in vivo into the central nervous system or into the ventricular cavities or subdurally onto the surface of a host brain. Such methods for grafting will be known to those skilled in the art and are described in Neural Grafting in the Mammalian CNS, Bjorklund and Stenevi, eds. (1985). Procedures include intraparenchymal transplantation, (i.e., within the host brain) achieved by injection or deposition of tissue within the host brain so as to be apposed to the brain parenchyma at the time of transplantation.

Administration of the cells or virus into selected regions of the recipient subject's brain may be made by drilling a hole and piercing the dura to permit the needle of a microsyringe to be inserted. The cells or recombinant lentivirus can alternatively be injected intrathecally into the spinal cord region. A cell preparation infected ex vivo, or the recombinant lentivirus of the invention, permits grafting of neuronal cells to any predetermined site in the brain or spinal cord, and allows multiple grafting simultaneously in several different sites using the same cell suspension or viral suspension and permits mixtures of cells from different anatomical regions.

Cells infected with a recombinant lentivirus of the invention, in vivo, or ex vivo, used for treatment of a neuronal disorder for example, may optionally contain an exogenous gene, for example, a gene which encodes a receptor or a gene which encodes a ligand. Such receptors include receptors which respond to dopamine, GABA, adrenaline, noradrenaline, serotonin, glutamate, acetylcholine and other neuropeptides, as described above. Examples of ligands which may provide a therapeutic effect in a neuronal disorder include dopamine, adrenaline, noradrenaline, acetylcholine, gamma-aminobutyric acid and serotonin. The diffusion and uptake of a required ligand after secretion by an infected donor cell would be beneficial in a disorder where the subject's neural cell is defective in the production of such a gene product. A cell genetically modified to secrete a neurotrophic factor, such as nerve growth factor (NGF), might be used to prevent degeneration of cholinergic neurons that might otherwise die without treatment. Alternatively, cells can be grafted into a subject with a disorder of the basal ganglia, such as Parkinson's disease, or can be modified to contain an exogenous gene encoding L-DOPA, the precursor to dopamine. Parkinson's disease is characterized by a loss of dopamine neurons in the substantia nigra of the midbrain, which have the basal ganglia as their major target organ.

Other neuronal disorders that can be treated similarly by the method of the invention include Alzheimer's disease, Huntington's disease, neuronal damage due to stroke, and damage in the spinal cord. Alzheimer's disease is characterized by degeneration of the cholinergic neurons of the basal forebrain. The neurotransmitter for these neurons is acetylcholine, which is necessary for their survival. Engraftment of cholinergic cells infected with a recombinant lentivirus of the invention containing an exogenous gene for a factor which would promote survival of these neurons can be accomplished by the method of the invention, as described. Following a stroke, there is selective loss of cells in the CA1 of the hippocampus as well as cortical cell loss which may underlie cognitive function and memory loss in these patients. Once identified, molecules responsible for CA1 cell death can be inhibited by the methods of this invention. For example, antisense sequences, or a gene encoding an antagonist can be transferred to a neuronal cell and implanted into the hippocampal region of the brain.

The method of transferring nucleic acid also contemplates the grafting of neuroblasts in combination with other therapeutic procedures useful in the treatment of disorders of the CNS. For example, the lentiviral infected cells can be co-administered with agents such as growth factors, gangliosides, antibiotics, neurotransmitters, neurohormones, toxins, neurite promoting molecules and antimetabolites and precursors of these molecules such as the precursor of dopamine, L-DOPA.

Further, there are a number of inherited neurologic diseases in which defective genes may be replaced including: lysosomal storage diseases such as those involving β-hexosamimidase or glucocerebrosidase; deficiencies in hypoxanthine phosphoribosyl transferase activity (the “Lesch-Nyhan” syndrome”); amyloid polyneuropathies (-prealbumin); Duchenne's muscular dystrophy, and retinoblastoma, for example.

For diseases due to deficiency of a protein product, gene transfer could introduce a normal gene into the affected tissues for replacement therapy, as well as to create animal models for the disease using antisense mutations. For example, it may be desirable to insert a Factor IX encoding nucleic acid into a lentivirus for infection of a muscle or liver cell.

Stem cell therapy contemplates injection of stem cells transduced by a lentiviral vector carrying a therapeutic gene of interest into a fetus central nervous system. The correction or rescue of a genetic defect is achieved during cell differentiation. Stem cells at a nondividing stage should be efficiently transduced by such a vector using a convenient infection technique.

The pharmacologically active compounds of this invention can be processed in accordance with conventional methods of galenic pharmacy to produce medicinal agents for administration to patients, e.g., mammals including humans.

The compounds of this invention can be employed in admixture with conventional excipients, i.e., pharmaceutically acceptable organic or inorganic carrier substances suitable for parenteral, enteral (e.g., oral) or topical application, which do not deleteriously react with the active compounds. Suitable pharmaceutically acceptable carriers include but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds. They can also be combined where desired with other active agents, e.g., vitamins.

For parenteral application, particularly suitable are injectable, sterile solutions, preferably oily or aqueous solutions, as well as suspensions, emulsions, or implants, including suppositories. Ampoules are convenient unit dosages.

For enteral application, particularly suitable are tablets, dragees, liquids, drops, suppositories, or capsules. A syrup, elixir, or the like can be used wherein a sweetened vehicle is employed.

Sustained or directed release compositions can be formulated, e.g., by inclusion in liposomes or those wherein the active compound is protected with differentially degradable coatings, e.g., by microencapsulation, multiple coatings, etc. It is also possible to freeze-dry these compounds and use the lyophilizates obtained, for example, for the preparation of products for injection.

For topical application, there are employed as non-sprayable forms, viscous to semi-solid or solid forms comprising a carrier compatible with topical application and having a dynamic viscosity preferably greater than water. Suitable formulations include but are not limited to solutions, suspensions, emulsions, creams, ointments, powders, liniments, salves, aerosols, etc., which are, if desired, sterilized or mixed with auxiliary agents, e.g., preservatives, stabilizers, wetting agents, buffers or salts for influencing osmotic pressure, etc. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., a freon.

It will be appreciated that the actual preferred amounts of active compound in a specific case will vary according to the specific compound being utilized, the compositions formulated, the mode of application, and the particular situs and organism being treated. Dosages for a given host can be determined using conventional considerations, e.g., by customary comparison of the differential activities of the subject compounds and of a known agent, e.g., by means of an appropriate, conventional pharmacological protocol.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Below are lists of viral promoters, cellular promoters/enhancers and inducible promoters/enhancers that could be used in combination with the present invention. Additionally any promoter/enhancer combination (as per the Eukaryotic Promoter Database, EPDB) could also be used to drive expression of exemplary constructs. Table 1 lists exemplary enhancer elements, while Table 2 lists examples of promoters.

TABLE 1

| Enhancer | References |
|---|---|
| Immunoglobulin Heavy Chain | Banerji et al., 1983; Gilles et al., 1983; Grosschedl and |
| | Baltimore, 1985; Atchinson and Perry, 1986, 1987; Imler |
| | et al., 1987; Weinberger et al., 1984; Kiledjian et al., |
| | 1988; Porton et al., 1990 |
| Immunoglobulin Light Chain | Queen and Baltimore, 1983; Picard and Schaffner, 1984 |
| T-Cell Receptor | Luria et al., 1987; Winoto and Baltimore, 1989; Redondo |
| | et al., 1990 |
| HLA DQα and DQβ | Sullivan and Peterlin, 1987; |
| β-Interferon | Goodbourn et al., 1986; Fujita et al., 1987; Goodbourn |
| | and Maniatis, 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al., 1989 |
| Muscle Creatine Kinase | Jaynes et al., 1988; Horlick and Benfield, 1989; Johnson |
| | et al., 1989a |
| Prealbumin (Transthyretin) | Costa et al., 1988 |
| Elastase I | Omitz et al., 1987 |
| Metallothionein | Karin et al., 1987; Culotta and Hamer, 1989 |
| Collagenase | Pinkert et al., 1987; Angel et al., 1987 |
| Albumin Gene | Pinkert et al., 1987; Tronche et al., 1989, 1990 |
| α-Fetoprotein | Godbout et al., 1988; Campere and Tilghman, 1989 |
| t-Globin | Bodine and Ley, 1987; Perez-Stable and Constantini, |
| | 1990 |
| β-Globin | Trudel and Constantini, 1987 |
| e-fos | Cohen et al., 1987 |
| c-HA-ras | Triesman, 1986; Deschamps et al., 1985 |
| Insulin | Edlund et al., 1985 |
| Neural Cell Adhesion | Hirsh et al., 1990 |
| Molecule (NCAM) | |
| α₁-Antitrypain | Latimer et al., 1990 |
| H2B (TH2B) Histone | Hwang et al., 1990 |
| Mouse or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A | Edbrooke et al., 1989 |
| (SAA) | |
| Troponin I (TN I) | Yutzey et al., 1989 |
| Platelet-Derived Growth | Pech et al., 1989 |
| Factor | |
| Duchenne Muscular | Klamut et al., 1990 |
| Dystrophy | |
| SV40 | Banerji et al., 1981; Moreau et al., 1981; Sleigh and |
| | Lockett, 1985; Firak and Subramanian, 1986; Herr and |
| | Clarke, 1986; Imbra and Karin, 1986; Kadesch and Berg, |
| | 1986; Wang and Calame, 1986; Ondek et al., 1987; Kuhl |
| | et al, 1987; Schaffner et al., 1988 |
| Polyoma | Swartzendruber and Lehman, 1975; Vasseur et al., 1980; |
| | Katinka et al., 1980, 1981; Tyndell et al., 1981; Dandolo |
| | et al., 1983; de Villiers et al., 1984; Hen et al., 1986; |
| | Satake et al., 1988; Campbell and Villarreal, 1988 |
| Retroviruses | Kriegler and Botchan, 1982, 1983; Levinson et al., 1982; |
| | Kriegler et al., 1983, 1984a, b, 1988; Bosze et al., 1986; |
| | Miksicek et al., 1986; Celander and Haseltine, 1987; |
| | Thiesen et al., 1988; Celander et al., 1988; Chol et al., |
| | 1988; Reisman and Rotter, 1989 |
| Papilloma Virus | Campo et al., 1983; Lusky et al., 1983; Spandidos and |
| | Wilkie, 1983; Spalholz et al., 1985; Lusky and Botchan, |
| | 1986; Cripe et al., 1987; Gloss et al., 1987; Hirochika et |
| | al, 1987; Stephens and Hentschel, 1987; Glue et al., 1988 |
| Hepatitis B Virus | Bulla and Siddiqui, 1986; Jameel and Siddiqui, 1986; |
| | Shaul and Ben-Levy, 1987; Spandau and Lee, 1988; |
| | Vannice and Levinson, 1988 |
| Human Immuno-deficiency | Muesing et al., 1987; Hauber and Cullan, 1988; |
| Virus | Jakobovits et al., 1988; Feng and Holland, 1988; Takebe |
| | et al., 1988; Rosen et al., 1988; Berkhout et al., 1989; |
| | Laspia et al., 1989; Sharp and Marciniak, 1989; |
| | Braddock et al., 1989 |
| Cytomegalovirus | Weber et al., 1984; Boshart et al., 1985; Foecking and |
| | Hofstetter, 1986 |
| Gibbon Ape Leukemia Virus | Holbrook et al., 1987; Quinn et al., 1989 |

TABLE 2

| Promoter Element | Inducer | References |
|---|---|---|
| MT II | Phorbol Ester (TFA) | Palmiter et al., 1982; |
| | Heavy metals | Haslinger and Karin, 1985; |
| | | Searle et al., 1985; Stuart et |
| | | al., 1985; Imagawa et al., |
| | | 1987, Karin et al., 1987; |
| | | Angel et al., 1987b; |
| | | MeNeall et al., 1989 |
| MMTV (mouse | Glucocorticoids | Huang et al., 1981; Lee et |
| mammary tumor | | al., 1981; Majors and |
| virus) | | Varmus, 1983; Chandler et |
| | | al., 1983; Lee et al., 1984; |
| | | Ponta et al., 1985; Sakai et |
| | | al., 1988 |
| β-Interferon | Poly(rI)x | Tavernier et al., 1983 |
| | poly(rc) | |
| Adenovirus 5 E2 | Ela | Imperiale and Nevins, 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, | |
| | Newcastle | |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene | Interferon | Blanar et al., 1989 |
| H-2Kb | | |
| HSP70 | Ela, SV40 Large T | Taylor et al., 1989; Taylor |
| | Antigen | and Kingston, 1990a, b |
| Proliferin | Phorbol Ester-TPA | Mordacq and Linzer, 1989 |
| Tumor Necrosis Factor | FMA | Hensel et al., 1989 |
| Thyroid Stimulating | Thyroid Hormone | Chatterjee et al., 1989 |
| Hormone a Gene | | |

A number of genes and proteins are contemplated for use in the therapeutic embodiments of the present invention. Below is a list of selected cloned structural genes that are contemplated for use in the present invention (Table 3). The list is not in any way meant to be interpreted as limiting, only as exemplary of the types of structural genes contemplated for use in the present invention. In addition, Table 4 below is an exemplary, but in no means limiting, list of proteins that may be used in the present invention.

TABLE 3

| | Selected Cloned Structural Genes | |
|---|---|---|
| Gene | Clone Type | Reference |
| Activin | porcine-cDNA | Mason A. J. 1985 Nature 318: 659 |
| Adenosine deaminase | h-cDNA | Wiginton D. A. 1983 PNAS 80: 7481 |
| Angiotensinogen I | r-cDNA | Ohkubo H. 1983 PNAS 80: 2196; |
| | r-gDNA | Tanaka T. 1984 JBC 259: 8063 |
| Antithrombin III | h-cDNA | Bock S. C. 1982 NAR 10: 8113; |
| | h-cDNA and | Prochownik E. V. 1983 JBC 258: 8389 |
| | gDNA | |
| Antitrypsin, alpha I | h-cDNA | Kurachi K. 1981 PNAS 78: 6826; Leicht |
| | h-gDNA RFLP | M. 1982 Nature 297: 655; Cox D. W. |
| | | 1984 AJHG 36: 134S |
| Apolipoprotein A-I | h-cDNA, h-gDNA | Shoulders C. C. 1982 NAR 10: 4873; |
| | RFLP | Karathanasis S. K. 1983 Nature |
| | h-gDNA | 301: 718; Kranthanasis S. K. 1983 PNAS |
| | | 80: 6147 |
| Apolipoprotein A-II | h-cDNA | Sharpe C. R. 1984 NAR 12: 3917; |
| | Chr | Sakaguchi A. Y. 1984 AJHB 36: 207S; |
| | h-cDNA | Knott T. J. 1984 BBRC 120: 734 |
| Apolipoprotein C-I | h-cDNA | Knott T. J. 1984 NAR 12: 3909 |
| Apolipoprotein C-II | h-cDNA | Jackson C. L. 1984 PNAS 81: 2945; |
| | h-cDNA | Mykelbost O. 1984 JBC 249: 4401; Fojo |
| | h-cDNA | S. S. 1984 PNAS 81: 6354; Humphries |
| | RFLP | S. E. 1984 C Gen 26: 389 |
| Apolipoprotein C-III | h-cDNA and | Karanthanasis S. K. 1983 Nature |
| | gDNA | 304: 371; Sharpe C. R. 1984 NAR |
| | h-cDNA | 12: 3917 |
| Apolipoprotein E | h-cDNA | Brewslow J. L. 1982 JBC 257: 14639 |
| Atrial natriuretic factor | h-cDNA | Oikawa S. 1984 Nature 309: 724; |
| | h-cDNA | Nakayama K. 1984 Nature 310: 699; |
| | h-cDNA | Zivin R. A. 1984 PNAS 81: 6325; |
| | h-gDNA | Seidman C.E. 1984 Sci 226: 1206; |
| | h-gDNA | Nemer M. 1984 Nature 312: 654; |
| | h-gDNA | Greenberg B. I. 1984 Nature 312: 665 |
| Chorionic gonadotropin, | h-cDNA | Fiddes J. C. 1981 Nature 281: 351; |
| alpha chain | RFLP | Boethby M. 1981 JBC, 256: 5121 |
| Chorionic gonadotropin, | h-cDNA | Fiddes J. C. 1980 Nature 286: 684; |
| beta chain | h-gDNA | Boorstein W. R. 1982 Nature 300: 419; |
| | h-gDNA | Talmadge K. 1984 Nature 307: 37 |
| Chymosin, pro (rennin) | bovine-cDNA | Harris T. J. R. 1982 NAR 10: 2177 |
| Complement, factor B | h-cDNA | Woods D. E. 1982 PNAS 79: 5661; |
| | h-cDNA and | Duncan R. 1983 PNAS 80: 4464 |
| | gDNA | |
| Complement C2 | h-cDNA | Bentley D. R. 1984 PNAS 81: 1212; |
| | h-gDNA (C2, C4, | Carroll M. C. 1984 Nature 307: 237 |
| | and B) | |
| Complement C3 | m-cDNA | Domdey H. 1983 PNAS 79: 7619; |
| | h-gDNA | Whitehead A. S. 1982 PNAS 79: 5021 |
| Complement C4 | h-cDNA and | Carroll M. C. 1983 PNAS, 80: 264; |
| | gDNA | Whitehead A. S. 1983 PNAS 80: 5387 |
| | h-cDNA | |
| Complement C9 | h-cDNA | DiScipio R. C. 1984 PNAS 81: 7298 |
| Corticotropin releasing | sheep-cDNA | Furutani Y. 1983 Nature 301: 537; |
| factor | h-gDNA | Shibahara S. 1983 EMBO J 2: 775 |
| Epidermal growth factor | m-cDNA | Gray A. 1983 Nature 303: 722; |
| | m-cDNA | Scott J. 1983 Sci 21: 236; Brissenden |
| | h-gDNA | J. E. 1984 Nature 310: 781 |
| Epidermal growth factor | h-cDNA and Chr | Lan C. R. 1984 Sci 224: 843 |
| receptor, oncogene c-erb | | |
| B | | |
| Epoxide dehydratase | r-cDNA | Gonzlalez F. J. 1981 JBC 256: 4697 |
| Erythropoietin | h-cDNA | Lee-Huang S. 1984 PNAS 81: 2708 |
| Esterase inhibitor, | h-cDNA | Stanley K. K. 1984 EMBO J 3: 1429 |
| dehydratase | | |
| Factor VIII | h-cDNA and | Gitschier J. 1984 Nature 312: 326; |
| | gDNA | Toole J. J. 1984 Nature 312: 342 |
| | h-cDNA | |
| Factor IX, | h-cDNA | Kutachi K. 1982 PNAS 79: 6461; Choo |
| Christmas factor | h-cDNA | K. H. 1982 Nature 299: 178; Camerino |
| | RFLP | G. 1984 PNAS 81: 498; Anson D. S. |
| | h-gDNA | 1984 EMBO J 3: 1053 |
| Factor X | h-cDNA | Leytus S. P. 1984 PNAS 81: 3699 |
| Fibrinogen A, alpha | h-cDNA | Kant J. A. 1983 PNAS 80: 3953 |
| B beta, gamma | h-gDNA (gamma) | Fornace A. J. 1984 Sci 224: 161; Imam |
| | h-cDNA (alpha | A. M. A. 1983 NAR 11: 7427; Fornace |
| | gamma) | A. J. 1984 JBC 259: 12826 |
| | h-gDNA (gamma) | |
| Gastrin releasing peptide | h-cDNA | Spindel E. R. 1984 PNAS 81: 5699 |
| Glucagon, prepro | hamster c-DNA | Bell G. I. 1983 Nature 302: 716; Bell |
| | h-gDNA | G. I. 1983 Nature 304: 368 |
| Growth hormone | h-cDNA | Martial J. A. 1979 Sci 205: 602; DeNoto |
| | h-gDNA | F. M. 1981 NAR 9: 3719; Owerbach D. |
| | GH-like gene | 1980 Sci 209: 289 |
| Growth hormone, RF | h-cDNA | Gubler V. 1983 PNAS 80: 3411 |
| Somatocrinin | h-cDNA | Mayo K. E. 1983 Nature 306: 86 |
| Hemopexin | h-cDNA | Stanley K. K. 1984 EMBO J 3: 1429 |
| Inhibin | porcine-cDNA | Mason A. J. 1985 Nature 318: 659 |
| Insulin, prepro | h-gDNA | Ullrich A. 1980 Sci 209: 612 |
| Insulin-like growth factor | h-cDNA | Jansen M. 1983 Nature 306: 609; Bell |
| I | h-cDNA | G. I. 1984 Nature 310: 775; Brissenden |
| | Chr | J. E. 1984 Nature 310: 781 |
| Insulin-like growth factor | h-cDNA | Bell G. I. 1984 Nature 310: 775; Dull |
| II | h-gDNA | T. J. 1984 Nature 310: 777; Brissenden |
| | Chr | J. E. 1984 Nature 310: 781 |
| Interferon, alpha | h-cDNA | Maeda S. 1980 PNAS 77: 7010; Goeddel |
| (leukocyte), multiple | h-cDNA | D. V. 1981 Nature 290: 20; Lawn R. M. |
| | h-gDNA | 1981 PNAS 78: 5435; Todokoro K. 1984 |
| | h-gDNA | EMBO J 3: 1809; Torczynski R. M. 1984 |
| | h-gDNA | PNAS 81: 6451 |
| Interferon, beta | h-cDNA | Taniguchi T. 1980 Gene 10: 11; Lawn |
| (fibroblast) | h-gDNA | R. M. 1981 NAR 9: 1045; Sehgal P. 1983 |
| | h-gDNA (related) | PNAS 80: 3632; Sagar A. D. 1984 Sci |
| | h-gDNA (related) | 223: 1312; Gray P. W. 1982 Nature |
| | h-cDNA | 295: 503 |
| Interferon, gamma | h-cDNA | Gray P. W. 1982 Nature 298: 859 |
| (immune) | h-gDNA | |
| Interleukin-1 | m-cDNA | Lomedico P. T. 1984 Nature 312: 458 |
| Interleukin-2, T-cell | h-cDNA | Devos R. 1983 NAR 11: 4307 |
| Growth factor | h-cDNA | Taniguchi T. 1983 Nature 302: 305; |
| | h-gDNA | Hollbrook N. J. 1984 PNAS 81: 1634; |
| | Chr | Siegel L. F. 1984 Sci 223: 175 |
| Interleukin-3 | m-cDNA | Fung M. C. 1984 Nature 307: 233 |
| Kininogen, two forms | bovine-cDNA | Nawa H. 1983 PNAS 80: 90; Kitamura |
| | bovine,-cDNA and | N. 1983 Nature 305: 545 |
| | gDNA | |
| Leuteinizing hormone, | h-gDNA and Chr | Talmadge K. 1984 Nature 207: 37 |
| beta subunit | | |
| Leuteinizing hormone | h-cDNA and | Seeburg P. H. 1984 Nature 311: 666 |
| releasing hormone | gDNA | |
| Lymphotoxin | h-cDNA and | Gray P. W. 1984 Nature 312: 721 |
| | gDNA | |
| Mast cell growth factor | m-cDNA | Yokoya T. 1984 PNAS 81: 1070 |
| Nerve growth factor, beta | m-cDNA | Scott J. 1983 Nature 302: 538; Ullrich |
| subunit | h-gDNA | A. 1983 Nature 303: 821; Franke C. |
| | Chr | 1983 Sci 222: 1248 |
| Oncogene, c-sis, PGDF | h-gDNA | Dalla-Favera R. 1981 Nature 295: 31 |
| Chain A | h-cDNA | Clarke M. F. 1984 Nature 208: 464 |
| Pancreatic polypeptide | h-cDNA | Boel E. 1984 EMBO J 3: 909 |
| and icosapeptide | | |
| Parathyroid hormone, | h-cDNA | Hendy G. N. 1981 PNAS 78: 7365; |
| prepro | h-gDNA | Vasicek T. J. 1983 PNAS 80: 2127 |
| Plasminogen | h-cDNA and | Malinowski D. P. 1983 Fed P 42: 1761 |
| | gDNA | |
| Plasminogen activator | h-cDNA | Edlund T. 1983 PNAS 80: 349; Pennica |
| | h-cDNA | D. 1983 Nature 301: 214; Ny T. 1984 |
| | h-gDNA | PNAS 81: 5355; Cook N. E. 1981 JBC |
| | h-cDNA | 256: 4007; Cooke N. E. 1982 Nature |
| | r-gDNA | 297: 603 |
| Prolactin | h-cDNA | Cook N. E. 1982 Nature 297: 603 |
| | r-gDNA | Cook N. E. 1981 JBC 256: 4007 |
| Proopiomelanocortin | h-cDNA | DeBold C. R. 1983 Sci 220: 721; Cochet |
| | h-gDNA | M. 1982 Nature 297: 335 |
| Protein C | h-cDNA | Foster D 1984 PNAS 81: 4766 |
| Prothrombin | bovine-cDNA | MacGillivray R. T. A. 1980 PNAS |
| | | 77: 5153 |
| Relaxin | h-gDNA | Hudson P. 1983 Nature 301: 628; |
| | h-cDNA (2 genes) | Hudson P. 1984 EMBO J 3: 2333; |
| | Chr | Crawford R. J. 1984 EMBO J 3: 2341 |
| Renin, prepro | h-cDNA | Imai T 1983 PNAS 80: 7405; Hobart |
| | h-gDNA | P. M. 1984 PNAS 81: 5026; Miyazaki H. |
| | h-gDNA | 1984 PNAS 81: 5999; Chirgwin J. M. |
| | Chr | 1984 SCMG 10: 415 |
| Somatostatin | h-cDNA | Shen I. P. 1982 PNAS 79: 4575; Naylot |
| | h-gDNA and Ri-IP | S. I. 1983 PNAS 80: 2686 |
| Substances P & K | bovine-gDNA | Nawa H. 1984 Nature 312: 729 |
| Tachykinin, prepro, | bovine-cDNA | Nawa H. 1983 Nature 306: 32 |
| Urokinase | h-cDNA | Verde P. 1984 PNAS 81: 4727 |
| Vasoactive intestinal | h-cDNA | Itoh N. 1983 Nature 304: 547 |
| peptide | | |
| Vasopressin | r-cDNA | Schmale H. 1983 EMBO J 2: 763 |

Key to Table 3: cDNA—complementary DNA; Chr—chromosome; gDNA—genomic DNA; RFLP—restriction fragment polymorphism; h—human; m—mouse, r—rat

TABLE 4
Heterologous Proteins for Use in the Present Invention

| Heterologous Protein | Reference |
|---|---|
| Human: | |
| 2AP-70 protein-tyrosine kinase | Isakov et al. 1996 |
| ABC transporter tap1 processing (TAP) | Meyer et al. 1994 |
| ABC transporter tap2 processing (TAP) | Meyer et al. 1994 |
| α₂ C₂ adrenoceptor | Marjamaki et al. 1994 |
| α-galactosidase A | Coppola et al. 1994 |
| α and β globins | Groebe et al. 1992 |
| α₁ glycine receptor | Cascio et al. 1993 |
| α-macroglobulins (αM) | Rompaey & Marynen 1992 |
| α and β platelet-derived growth factor receptors | Jensen et al. 1992 |
| Adenosine deaminase | Medin et al. 1990 |
| aldase reductase | Nishimura et al. 1991 |
| α-interferon | Maeda et al. 1985 |
| 5-α reductase (type 1) | Delos et al. 1994 |
| Ah receptor and Ah receptor nuclear translocater | Chan et al. 1994 |
| Alzheimer amyloid precursor protein | Ramakrishna et al. 1991 |
| Alzheimer β-amyloid peptide precursor | Currie et al. 1991 |
| Amyloid peptide precursor | Essalmani et al. 1996 |
| Amyloid precursor protein | Bhasin et al. 1991 |
| Amyloid β protein precursor | Bhasin et al. 1991 |
| Amyloid precursor protein | Lowery et al. 1991 |
| Androgen receptor | Beitel et al. 1995 |
| Angiotensin | Williams et al. 1994 |
| Androgen receptor | Chang et al. 1992 |
| Antithrombin III | Gillespie et al. 1991 |
| Apolipoprotein E | Gretch et al. 1991 |
| Aromatase P450 | Amarneh & Simpson 1995 |
| Autoantigen of Wegener's granulomatosis (PR3) | Szymkowiak et al. 1996 |
| b1,2-N-acetylglucosaminyl-transferase I (hGNT-I) | Wagner et al. 1996 |
| β₁γ₂ dimers of G-protein | Dietrich et al. 1992 |
| β₁,β₂,γ₂ subunits of hetertrimeric guanine nucleotide- | Graber et al. 1992 |
| binding protein | |
| β₁-adrenergic receptor | Ravet et al. 1992 |
| β₂-adrenergic receptor | Kleymann et al. 1993 |
| β-adrenergic receptor kinase | Sohlemann et al. 1993 |
| β galactosidase | Itoh et al. 1990 |
| β interferon | Smith et al. 1983 |
| β2-glycoprotein I | Igarashi et al. 1996 |
| BCl2 | Alnemri et al. 1992 |
| BCl-2 oncoprotein | Reid et al. 1992 |
| Bone morphogenetic protein-2 | Maruoka et al. 1995 |
| Cc gene | Poul et al. 1995 |
| Cg1 sequence | Poul et al. 1995 |
| C-reactive protein | Marnell et al. 1995 |
| cAMP-specific phosphodiesterase | Amegadzie et al. 1995 |
| CD95/APO-1/Fas ligand | Mariani et al. 1996 |
| CD4 | Murphy et al. 1990; Lazarte et |
| | al. 1992 |
| Cdc42 GTP-binding protein | Cerione et al. 1995 |
| c-fos protein | Trainer et al. 1990 |
| CYP2A6 | Nanji et al. 1994 |
| Calpain I | Meyer et al. 1996 |
| Carcinoembryonic antigen | Bei et al. 1994 |
| Carcinoembryonic antigen CD 66b | Yamamaka et al. 1996 |
| Carcinoembryonic antigen CD66c | Yamamaka et al. 1996 |
| Cholecystokinin B (CCK_{B}) | Gimpl et al. 1996 |
| Choriogonadotropin α subunit | Nakhai et al. 1991 |
| Choriogonadotropin β-subunit | Chen et al. 1991 |
| Choriogonadotropin β-subunit descarboxyl-terminal | Chen and Bahl 1991 |
| peptide | |
| Chorionic gonadotropin hormone precursor | Nakhai et al. 1991 |
| Chorionic gonadotropin hormone (β-subunit) | Hasnain et al. 1994 |
| Chorionic gonadotropin hormone β subunit | Nakhai et al. 1992 |
| Complement Clr | Sass et al. 19?? |
| Complement Clr proenzyme | Gal et al. 1989 |
| Complement protein C9 | Tomlinson et al. 1993 |
| Corticosteroid binding globulin | Ghose Dastidar et al. 1991 |
| c-myc protein | Miyamoto et al. 1985 |
| Complement protein C9 | Tomlinson et al. 1993 |
| Corticosteroid binding globulin (hCBG) | Ghose-Dastidar et al. 1991 |
| Creatine kinase B (B-CK) | de Kok et al. 1995 |
| Cyclooxygenase-2 | Cromlish et al. 1994 |
| Cytochrome b₅ | Patten & Koch 1995 |
| Cytochrome B₅₅₈ | Katkin et al. 1992 |
| Cytochrome CYP3A4 | Lee et al. 1995 |
| Cytochrome P450 CYP3A4 | Buters et al. 1994 |
| Cytochrome P-450 isoform(s) | Claire et al. 1994 |
| Cytomegalovirus 65K tegument phosphoprotein | La Fauci et al. 1994 |
| Cytomegalo virus IE1, IE1 exon 4 | Davrinche et al. 1993 |
| Cytosolic phospholipase A₂ | Abdullah et al. 1995 |
| D4 dopamine receptor | Mills et al. 1993 |
| DNA ligase I | Gallina et al. 1995 |
| DNA polymerase α subunit | Copeland and Wang 1991 |
| DNA polymerase d catalytic subunit | Zhou et al. 1996 |
| DNA topoisomerase 1 | Zhelkovsky & Moore 1994 |
| Dopamine D2 receptor | Javitch et al. 1994 |
| EGF receptor | Greenfield et al. 1988 |
| EGF receptor-tyrosine kinase domain | Wedegaertner et al. 1989 |
| Endothelial nitric oxide synthase | Chen et al. 1996 |
| Epidermal growth factor receptor | Waterfield & Greenfield 1991 |
| Epidermal-growth-factor receptor protein-tyrosine kinase | McGlynn et al. 1992 |
| Epidermal growth factors IX and XIIa | Astermark et al. 1994 |
| Erythrocyte anion exchanger | Dale et al. 1996 |
| Erythropoietin | Quelle et al. 1992 |
| Estrogen receptor | Beekman et al. 1994 |
| Factor VIII - B domain deleted | Webb et al. 1993 |
| Fibroblast growth factor receptor subtype ligand binding | Sisk et al. 1992 |
| domain | |
| Follicle-stimulating hormone receptor | Christophe et al 1993 |
| Furin | Bravo et al. 1994 |
| GABA_{A} receptor α1 subunits | Birnir et al. 1995 |
| GABA_{A} receptor β1 subunits | Birnir et al. 1995 |
| ga773 - 2 antigen | Strassburg et al. 1992 |
| GMP synthetase | Lou et al. 1995 |
| Glucocerebrosidase | Martin et al. 1988 |
| Glucocorticoid receptor | Srinivasan et al. 1990 |
| Glutamic acid decarboxylase | Mauch et al. 1993 |
| Glycine receptor α1 | Morr et al. 1995 |
| Group b rotavirus ADRV, VP4 | Mackow et al. 1993 |
| Group II Phospholipase A2 | Tremblay et al. 1993 |
| Growth hormone | Sumathy et al. 1996 |
| Growth hormone receptor - extracellular domain | Ota et al. 1991 |
| 5-HT_{1A} receptor | Mulheron et al. 1994 |
| hst-1 transforming protein | Miyagawa et al. 1988 |
| Heart (R)-3-hydroxybutyrate dehydrogenase | Green et al. 1996 |
| Hematopoietic glycopeptide erythropoietin | Quelle et al. 1992 |
| Hemopexin | Satoh et al. 1994 |
| Heparin cofactor II | Ciaccia et al. 1995 |
| Hepatitis b virus X protein | Klein et al. 1992 |
| Hepatocyte growth factor | Yee et al. 1993 |
| Hepatocyte growth factor | Lee et al. 1993 |
| High-affinity IgE receptor-a chain | Yagi et al. 1994 |
| 17b-hydroxysteroid dehydrogenase | Breton et al. 1994 |
| 5-hydroxytryptamine_{1A} | Butkerait et al. 1995 |
| 5-hydroxytryptamine receptors (5-HT_{1A}, 5-HT_{1Dα}, 5- | Parker et al. 1994 |
| HT_{1Dβ}, 5-HT_{1E}) | |
| IgA | Carayannopoulos et al. 1994 |
| IL2 receptor α & β chains | Lindqvist et al 1993 |
| Immunodeficiency virus-type 1 gag precursor | Chazal et al. 1994 |
| Immunodeficiency virus-1 gp41 | Lu et al. 1993 |
| Immunodeficiency virus-1 gp120 | Yeh et al. 1993 |
| Insulin holoreceptor | Paul et al. 1990 |
| Insulin receptor substrate-1 | Siemeister et al. 1995 |
| Insulin receptor β-subunit | Herrera et al. 1988 |
| Insulin receptor β subunit transmembrane/cytoplasmic | Li et al. 1992 |
| domain | |
| Insulin receptor ectodomain | Sissom et al. 1989; 1991 |
| Insulin receptor protein-tyrosine kinase domain | Ellis et al. 1988 |
| Insulin receptor cytoplasmic domain of β subunit | Herrera et al. 1988 |
| Insulin receptor protein tyrosine-kinase-cytoplasmic | Ellis and Levine 1991 |
| domain | |
| Insulin-like growth factor II | Congote and Li, 1994 |
| Insulin-like growth factor II | Marumoto et al. 1992 |
| Intercellular adhesion molecule 1 (ICAM-1) | Cobb et al. 1992 |
| Interferon-g glycoforms | Ogonah et al. 1995 |
| Interleukin 2 | Smith et al. 1985 |
| Interleukin 2 glycoprotein variants | Grabenhorst et al. 1993 |
| Interleukin-2 receptor gamma chain | Raivio et al. 1995 |
| Interleukin 5 | Brown et al. 1995 |
| Interleukin 6 | Matsuura et al. 1991 |
| Interleukin-6 receptor | Weiergraber et al. 1995 |
| Intrinsic factor | Gordon et al. 1992 |
| Iron regulatory factor | Emery-Goodman et al. 1993 |
| Isoforms (neuronal, inducible, endothelial) nitric oxide | Nakane et al. 1995 |
| synthase | |
| Ku autoantigen | Allaway et al. 1990 |
| Lecithin-cholesterol acyltransferase | Chawla & Owen 1995 |
| Leukotriene A₄ hydrolase | Gierse et al. 1993 |
| Link protein | Grover & Roughley 1994 |
| Liver carboxylesterase | Kroetz et al. 1993 |
| Lymphocytic activation gene (LAG-1) | Baizleras et al 1990 |
| Lysyl hydroxylase | Krol et al. 1996; Pirskanen et |
| | al. 1996 |
| Lysosomal β-galactosidase | Itoh et al. 1991 |
| 5′lipoxygenase | Dunk et al. 1989 |
| μ1 muscarinic acetylcholine receptors | Haga et al. 1996 |
| μ2 muscarinic cholinergic receptor | Debburman et al. 1995 |
| μ3 (hμ3) muscarinic cholinergic receptors | Debburman et al. 1995 |
| MHC class I HLA-b27 antigen | Levy and Kvist 1990 |
| MHC class II DR4a, DR4b, extracellular domain | Scheerle et al. 1992 |
| Macrophage colony stimulating factor | Qiu et al. 1995 |
| Matrilysin | Lopez de Turiso et al. 1996 |
| Metallothionein-II | Schmiel et al. 1985 |
| Mineralocorticosteriod receptor | Binart et al. 1991 |
| Monocyte chemoattractant protein-1 | Ueda et al. 1994; Ishii et al. |
| | 1995 |
| Multidrug resistance 1 | Germann et al. 1990 |
| Multidrug resistance P-glycoprotein | Rao et al. 1994 |
| Muscarine receptor μ2 | Kameyama et al. 1994 |
| Myeloperoxidase | Taylor et al. 1992 |
| Myogenic factors myf4, myf5 | Braun et al. 1991 |
| N-formyl peptide receptor | Quehenberger et al. 1992 |
| Na⁺/H⁺ antiporter | Fafournoux et al. 1991 |
| NADPH-P450 oxidoreductase | Tamura et al. 1992 |
| Nerve growth factor | Buxser et al. 1991 |
| Nerve growth factor receptor | Vissavajjhala et al. 1990 |
| Neutrophil NADPH oxidase factors p47-[phox], | Leto et al. 1991 |
| p67[phox] | |
| Nuclear hormone receptor H-2R11BP | Marks et al. 1992 |
| Nucleolar protein p120 | Ren et al. 1996 |
| Oxytocin receptor | Gimpl et al. 1995 |
| p53 | Patterson et al. 1996 |
| P450 2E1 | Patten & Koch 1995 |
| Pancreatic lipase | Thirstrup et al. 1993 |
| Pancreatic procolipase | Lowe 1994 |
| Papillomavirus type 11 E1, E2 | Bream et al. 1993 |
| Papillomavirus type 11 L1 protein | Rose et al. 1993 |
| Papillomavirus type 16 E2 | Sanders et al. 1995 |
| Papillomavirus type 16 E2 protein | Kirnbauer et al. 1993 |
| Papillomavirus type 45 L1 protein | Touze et al. 1996 |
| Parainfluenza virus type 3, 7, HN, 7HN | Lehman et al. 1993 |
| Parathyroid hormone | Mathavan et al. 1995 |
| Parvovirus B19 vp1, vp2 | Cubie et al. 1993 |
| Phospholipase A₂ | Abdullah et al. 1995 |
| Placental aromatase (CYP19A1) | Sigle et al. 1994 |
| Plasma plasminogen | Whitefleet-Smith et al. 1989 |
| Plasminogen | Davidson et al. 1991 |
| Plasminogen (HPg) | Castellino et al. 1993 |
| Plasminogen activator inhibitor-2 | Pei et al. 1995 |
| Platelet glycoprotein IBb | Finch et al. 1996 |
| Platelet 12-lipoxygenase | Chen et al. 1993 |
| Poly(ADP-ribose) polymerase | Giner et al. 1992 |
| Pre-pro endothelin-1 | Benatti et al. 1992 |
| Pre-pro gastrin releasing peptide | Lebacq-Verheyden et al. 1988 |
| Pro-al(III) chains | Tomita et al. 1995 |
| ProapoA-I | Sorci-Thomas et al. 1996 |
| Progesterone receptor (A form) | Elliston et al. 1992 |
| Progesterone receptors A&B forms | Christensen et al. 1991 |
| Prolyl 4-hydroxylase a, b subunits | Vuori et al. 1992 |
| Prolyl 4-hydroxylase a subunit with BiP polypeptide | Veijola et al. 1996 |
| Prosaposin | Leonova et al. 1996 |
| Prostaglandin G/H synthase | George et al. 1996 |
| Prostaglandin G/H synthase 1 | Barnett et al. 1994 |
| Prostaglandin G/H synthase 2 | Barnett et al. 1994 |
| Protein disulphide isomerase | Vuori et al. 1992 |
| Protein kinase c-d | Rankl et al. 1994 |
| Protein kinase Cm | Dieterich et al. 1996 |
| Pro-urokinase | Gao and Hu 1994 |
| rab 6 | Yang et al. 1992 |
| rap1A | Quilliam et al. 1990 |
| Recombinant IL-8 | Kang et al. 1992 |
| Recombinant p56^{1ck} | Flotow et al. 1996 |
| Renin | Mathews et al. 1996 |
| Respiratory syncytial virus F and G glycoproteins | Wathen et al. 1989 |
| Retinoblastoma pp110^{RB} | Wang et al. 1990 |
| Retinoic acid receptor a1 | Quick et al. 1994 |
| Retinoic acid receptor - g1 | Reddy et al. 1992 |
| ssDNA-binding protein | Stigger et al. 1994 |
| Sex steroid-binding protein (hSBP/hABP, hSHBG) | Sui et al. 1995 |
| Soluble human insulin receptor - ectodomain | Sissom and Ellis 1992 |
| Soluble human insulin receptor tyrosine kinase | Ahn et al. 1993 |
| Sos1 protein | Frech et al. 1995 |
| Steroid 5a-reductase | Iehle et al. 1993 |
| Synthetic basic fibroblast growth factor | Hills & Crane-Robinson 1995 |
| TII (CD2) t-lymphocyte surface glycoprotein | Richardson et al. 1988 |
| TII (CD2) | Alcover et al. 1988 |
| T-cell leukemia virus type I p40 | Nyunoya et al. 1988 |
| T-cell protein tyrosine kinase | Lehr et al. 1996 |
| T-cell protein-tyrosine-phosphatase | Zander et al. 1991 |
| T-lymphotropic virus type 1 envelope protein | Yamashita et al. 1992 |
| Terminal transferase | Chang et al. 1988 |
| Terminal deoxynucleotidyl transferase | di Primio et al. 1992 |
| Thrombomodulin | Marumoto et al. 1993 |
| Thromboxane synthase | Yokoyama et al. 1993 |
| Thyroid hormone B₁ receptor | Putlitz et al. 1991 |
| Thyroid peroxidase | Kendler et al. 1993 |
| Thyrotropin receptor extracellular domain | Seetharamaiah et al. 1993 |
| Thyrotropin hormone receptor - extracellular domain | Huang et al. 1993 |
| Tissue inhibitor of metalloproteinases-1 | Gomez et al. 1994 |
| Tissue plasminogen activator | Jarvis et al. 1993 |
| Tissue-type plasminogen activator | Steiner et al. 1988 |
| Trancobalamin II isoproteins | Quadros et al. 1993 |
| Tyrosine hydroxylase | Ginns et al. 1988 |
| Tryptase | Sakai et al. 1996 |
| Tumor necrosis factor-b | Chai et al. 1996 |
| Type II collagen | Lamberg et al. 1996 |
| Urokinase | Laurie et al. 1995 |
| Urokinase-type plasminogen activator | King et al. 1991 |
| Vascular cell adhesion molecule-1 | Stoltenborg et al. 1993 |
| Vascular cell adhesion molecule-1 (VCAM1) | Stoltenborg et al. 1994 |
| Vascular endothelial growth factor VEGF₁₂₁, VEGF₁₆₅ | Fiebich et al. 1993 |
| Vitamin D receptor | Nakajima et al. 1993 |
| Vitronectin | Zhao and Sane, 1993 |
| Y1 neuropeptide Y receptor | Munoz et al. 1995 |
| Yoked chorionic gonadotropin | Narayan et al. 1995 |
| Hyalophora cecropia pupae attacin | Gunn et al. 1990 |

### Construction of Lentiviral Vectors Encoding VP22-EGFP Fusion Protein

We previously designed pseudotyped, high-titer, replication-defective HIV-1 vector systems to deliver genes into nondividing cells (Reiser, J. et al. 1996 PNAS USA 93:15266-15271). In the present study, we constructed double-gene lentiviral vectors encoding EGFP driven by the human CMV (cytomegalovirus)-IE (immediate early) promoter and the murine HSA driven by the viral long terminal repeat (LTR). One of the vector constructs (HIV-VP22-EGFP/HSA) encodes EGFP fused at its N terminus to the VP22 coding region (15) (FIG. 1B, Lower). A control vector (HIV-EGFP/HSA) (FIG. 1B Upper) expresses unfused EGFP. A three-plasmid expression system consisting of a defective packaging construct (FIG. 1A), a plasmid coding for the vesicular stomatitis virus (VSV) G glycoprotein (FIG. 1C), and the vector constructs shown in FIG. 1B were used to generate pseudotyped HIV-1 particles by transient transfection of human embryonic kidney 293T cells.

### Analysis of Cells Transduced with Double-Gene Vectors Encoding VP22-EGFP Fusion Protein

Double-gene vectors encoding EGFP (enhanced green fluorescent protein) and HSA (heat-stable antigen) were initially designed to distinguish recipient cells that have taken up the VP22 fusion protein from infected cells delivering the fusion protein. HOS cells infected with these vectors were EGFP-positive as well as HSA-positive by FACS (fluorescence-activated cells sorter) analysis. However, FACS analyses and Northern-blot assay revealed that the number of HSA-positive HOS cells infected with the HIV-VP22-EGFP/HSA vector was notably lower than the number of HSA-positive cells obtained from cultures infected by using HIV-EGFP/HSA vector system. These results imply that VP22 somehow affected HSA expression, possibly by down-regulating HSA-specific RNAs.

To rule out pseudotransduction events, we prepared vector stocks lacking a viral envelope glycoprotein (Env). FACS analysis revealed that a significant number of EGFP-positive cells were evident in cultures infected by lentiviral vector of HIV-VP22-EGFP/HSA, but not for cells infected by the HIV-VP22-EGFP/HSA lacking Env. Thus, the transport function of VP22-EGFP fusion protein was abolished when cells were transduced with viral stock lacking an envelope.

### Intercellular Spread of VP22-EGFP Fusion Proteins from Lentivirus-Transduced Cells

Because the VP22 fusion protein down-regulated the expression of HSA, we adopted a more indirect strategy previously introduced by Elliott and O'Hare (Elliott, G. & O'Hare, P. 1997 Cell 88:223-233) for transfected cells. To visualize transduction events involving the movement of VP22-EGFP to neighboring cells, COS-7 cells expressing simian virus 40 T-antigen were infected with the double-gene lentiviral vectors as described above. At 24 h after infection, the cells were coplated with a number of different types of uninfected cells at a ratio of 1:10. The expression of VP22-EGFP fusion proteins in transduced COS-7 cells and the spread of such proteins to uninfected cells, including brain choroid plexus cells, human endothelial cells, and HeLa cells, was investigated by fluorescence microscopy. The microscopic analysis indicated the transfer of VP22-EGFP into neighboring brain choroid plexus cells and human endothelial cells. Furthermore, VP22 fusion proteins in coplated human endothelial cells and HeLa cells were found in both the nucleus and the cytoplasm. The infected COS-7 cells were distinguished from uninfected cells by a monoclonal antibody specific for simian virus 40 T-antigen conjugated to tetramethylrhodamine isothiocyanate. The ratio of infected cells to neighboring recipient cells was as follows: 1.3±0.33 to 8.3±0.23 (P<0.05) for brain choroid plexus cells; 2.0±1.0 to 11.6±2.6 (P<0.05) for human endothelial cells, and 1.7±0.3 to 10.6±3.0 (P<0.05) for HeLa cells. The increases in the number of EGFP-positive recipient cells were significant in all three cell lines compared with the number of transduced delivery cells (P<0.05). EGFP was not transported to neighboring cells when COS-7 cells were infected with an HIV-EGFP/HSA vector lacking the VP22 coding sequence.

To demonstrate the specificity of VP22-EGFP protein transfer more directly, human H9 cells were used. H9 cells express IL-2Rs (Gazdar, A. F. et al. 1980 Blood 55:409-417). These surface receptors can be directly detected by an IL-2R-specific monoclonal antibody. We transferred H9 suspension cells into a culture dish on which transduced COS-7 cells had already adhered and grown for 24 h. Nonadherent cells were removed 3 d later and subjected to fluorescence microscopy. The microscopic analysis indicated that not only did the H9 cells exhibit binding of a phycoerythrin-labeled IL-2R-specific monoclonal antibody, but a significant number of those cells also displayed green fluorescence. H9 cells cocultured with COS-7 cells previously infected by the HIV-EGFP/HSA vector displayed red fluorescence but the green fluorescence was greatly reduced and there were no doubly positive cells. The results support the hypothesis that the green fluorescence in H9 cells resulted from the transfer of EGFP mediated by VP22 from the COS-7 cells.

### Lentiviral Vector Delivery of VP22-EGFP Fusion Protein in Mouse Brain

To determine the capacity to deliver VP22-EGFP from lentivirus-transduced cells in vivo, H9 cells previously infected by lentiviral vectors were injected into the ventricles of brains of mice. The results indicated that VP22-EGFP fusion protein had spread into the neighboring tissues from the ventricle, and even as far as the cerebral cortex. However, we did not observe such significant transport of EGFP into neighboring tissues, nor the cortex when the implanted cells were previously transduced with the HIV-EGFP lentiviral vector lacking VP22. The transplanted H9 cells in brain ventricles were detected by a specific IL-2R antibody.

VP22-EGFP in the cortical region of the mouse brain was observed not only in the nuclei of cortical cells, but also in the cytoplasm of axons.

To further study the delivery of VP22-EGFP fusion protein by lentiviral vector in mouse brain, we injected the viral vectors directly into the pyramidal cell layer in area CA2 of the hippocampus. VP22-EGFP fusion protein was transported throughout the whole pyramidal cell and oriens layers of the hippocampus. Only a local diffusion of EGFP was found when HIV-EGFP/HSA vector lacking a VP22 coding sequence was injected.

### Design of Multigene HIV-1-Based Vector Systems

We previously described two different classes of HIV-1-based gene transfer vectors encoding single reporter genes such as EGFP, HSA, and ShlacZ and the application of such vectors to deliver reporter genes into nondividing cells (Mochizuki, H. et al. 1998 J Virol 72:8873-8883). These vectors also contained cis-acting sequences required for packaging, reverse transcription, and integration, including the 5′ and 3′ LTRs, and Env-derived sequences encompassing the Rev-responsive element (RRE). One class of vectors was defective for all HIV-1 genes but encoded functional Tat and Rev with the transgene placed within the env coding region 5′ to the RRE. Vectors lacking Tat and Rev with the expression cassette located 3′ to the RRE were also constructed in accordance with the design of Parolin et al. (Parolin, C. et al. 1996 Virology 222:415-422) and Naldini et al. (Naldini, L. et al. 1996 Science 272:263-267). We have now modified these vectors for the concurrent expression of multiple transgenes. Single-gene vectors, bicistronic vectors, or multigene vectors able to express up to three exogenous genes under the control of two or three different transcriptional units placed within the viral gag-pol coding region and/or the viral nef and env genes were designed (FIG. 2). The genes encoding EGFP, HSA, a cell surface marker, and bacterial neomycin phosphotransferase (Neo) were used as models whose expression was monitored by FACS, fluorescence microscopy, and G418 selection. The additional components of the gene transfer system include a packaging (helper) plasmid and an envelope (Env) plasmid encoding VSV-G driven by the HIV-1 LTR (Mochizuki, H. et al. 1998 J Virol 72:8873-8883; Reiser, J. et al. 1996 PNAS USA 93:15266-15271). Pseudotyped vectors were produced in human embryonic kidney 293T cells using a three-component transient packaging system (Mochizuki, H. et al. 1998 J Virol 72:8873-8883).

Multigene Vectors Involving Two Separate Transcriptional Units

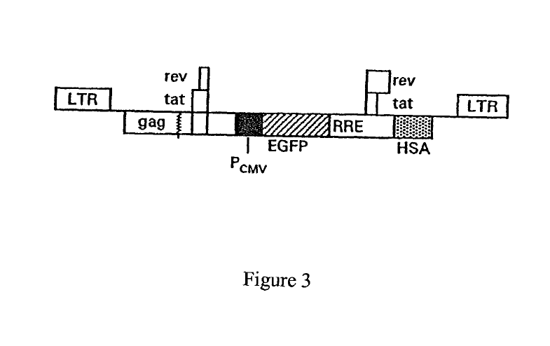

With a view toward-designing vectors that are useful in anti-HIV and other gene therapy strategies, HIV-1-based vectors with the potential to coexpress multiple transgenes as separate transcriptional units were designed. To construct a two-gene vector expressing two separate genes from two independent promoters, the original HIV-EGFPΔE vector (Mochizuki, H. et al. 1998 J Virol 72:8873-8883) containing the EGFP reporter gene linked to the CMV IE promoter was engineered to express the HSA cell surface marker. To generate the two-gene HIV-EGFP-HSAΔE vector (FIG. 3), the nef coding region was replaced with the mouse HSA cDNA. In this construct, a functional tat-coding region was retained, allowing expression of gene sequences placed within the nef-coding region from a multiply spliced mRNA through activation of the viral LTR. Fluorescence-activated cell sorting and fluorescence microscopy indicated that coexpression of the EGFP and HSA genes in dividing and nondividing cells was achieved.

### Multigene Vectors Involving Three Separate Transcriptional Units

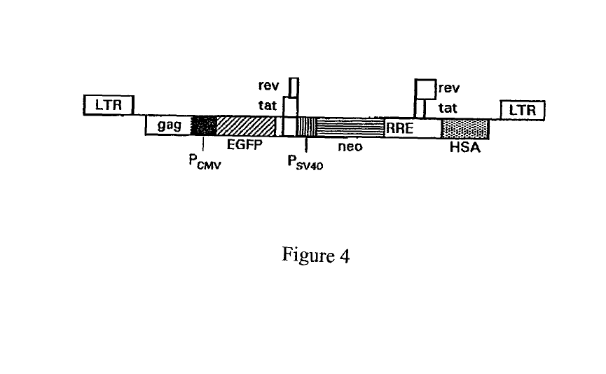

To investigate the potential to express three independent transcriptional units in the context of a Tat-containing lentivirus vector, a construct coexpressing three different transgenes under the control of three separate promoters was designed (FIG. 4). In this vector, the CMV IE promoter and EGFP gene were placed within the viral gag-pol-coding region. The env gene was deleted to accommodate the bacterial neo gene driven by the SV40 early promoter, and the HSA gene was placed within the nef-coding region. Fluorescence-activated cell sorting and fluorescence microscopy indicated that coexpression of the EGFP and HSA genes in G418 selected dividing and nondividing cells was achieved.

### Expression from Bicistronic Vectors

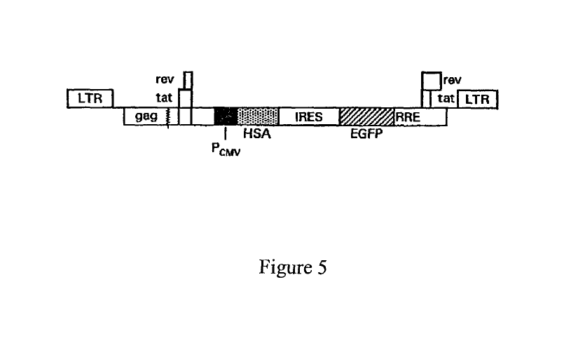

Bicistronic vectors rely on a single promoter driving two or more separate protein coding regions linked by internal ribosome entry site (IRES) sequences. Cassettes carrying HSA and EGFP genes linked by IRES sequences in one transcriptional unit were designed and introduced into two different HIV-1-based vector backbones. A vector (HIV-HAS-IRES-EGFPΔE) containing the ECMV IRES with functional tat and rev coding regions and the bicistronic expression cassette placed 5′ to the RRE was constructed first (FIG. 5). Fluorescence-activated cell sorting indicated that coexpression of the EGFP and HSA genes in representative cells was achieved.

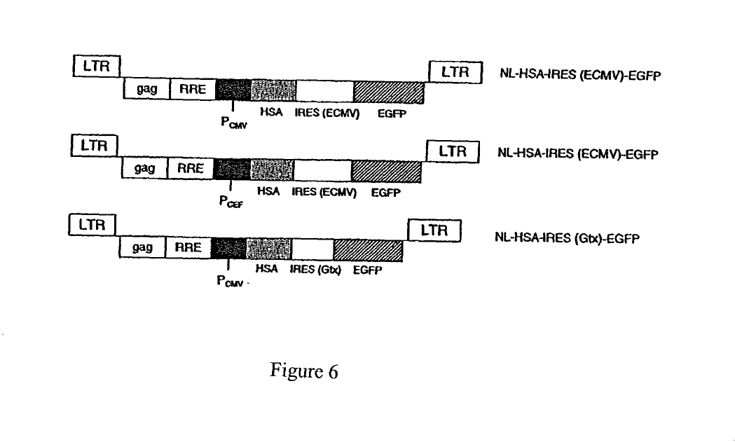

Bicistronic vectors lacking Tat and Rev with the expression cassette loaded 3′ to the RRE were designed next (FIG. 6). The ECMV IRES was used along with the homeobox-derived Gtx IRES to yield NL-HSA-IRES (ECMV)-EGFP and NL-HSA-IRES (Gtx)-EGFP, respectively. FACS analysis indicated that both vectors yielded doubly positive cells. The NL-HSA-IRES (ECMV)-EGFP/CEP vector construct harboring the CEF promoter in place of the CMV IE promoter also produced doubly positive cells. The results indicated that expression of the EGFP cistron was strongly affected by the promoter used and by the IRES sequence.

### Lentiviral Injections into the Striatum

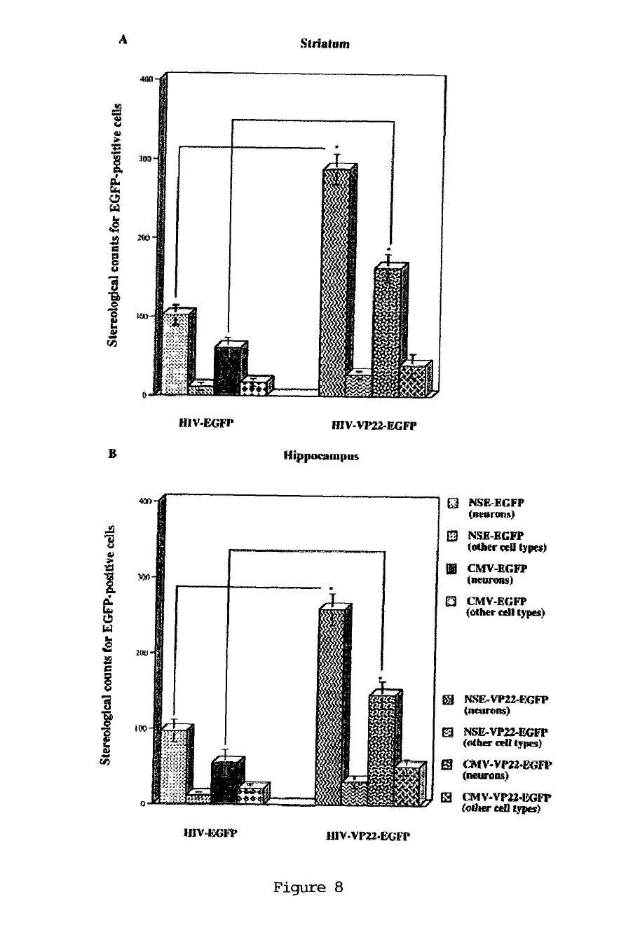

Lentiviral vectors (FIG. 7) were injected directly into the nucleus accumbens in the striatum of mice brains. Mice were sacrificed 3 months postinjection. Transduced cells in the striatum displayed extensive EGFP. The EGFP-positive cells in the brain sections were examined by confocal microscopy and immunofluorescence assay. Brain sections showed a large number of EGFP-positive cells transduced by the lentiviral vector HIV-NSE-VP22-EGFP from the injection site as compared with the number of EGFP-positive cells transduced by the HIV-NSE-EGFP. The same pattern of EGFP distribution in the striatum and hippocampus in mice brain injected with the lentiviral vectors with or without VP22 driven by the CMV promoters were also observed with confocal microscopy. Stereological counts of EGFP-positive cells in the CNS were performed on the brain slides by scanning with a laser confocal microscope. In the mouse striatum, a total of 315±27 EGFP-positive cells per slide were present in the mice brains (n=5) injected with HIV-NSE-VP22-EGFP and 113±15 EGFP-positive cells were found per slide in brain sections of those injected with HIV-NSE-EGFP. A total number of 202±21 EGFP-positive cells for HIV-CMV-VP22-EGFP and 78±7.0 EGFP-positive cells for HIV-CMV-EGFP, respectively, were also recorded per slide in the mice brains. The data indicated that the EGFP-positive cells for both NSE-VP22-EGFP and CMV-VP22-EGFP are significantly higher than the numbers of those injections for NSE-EGFP and CMV-EGFP lentiviral vectors without VP22 (FIG. 8A)

### Lentiviral Injection into the Hippocampus

To further study the delivery of VP22-EGFP fusion protein by lentiviral vector in mouse brain, we injected the viral vectors directly into the pyramidal cell layer (CA2 area) of the hippocampus. VP22-EGFP fusion protein was transported throughout the entire pyramidal cell and oriens layers of the hippocampus injected with HIV-NSE-VP22-EGFP. Only a local diffusion of EGFP was found when HIV-NSE-EGFP vector lacking a VP22 coding sequence was injected. Stereological counts of EGFP-positive cells in the hippocampus were also performed on the brain slides scanned with the laser confocal microscopy. In the mouse hippocampus, a total of 290±20 EGFP-positive cells per slide were counted in brain sections (n=5) injected with HIV-NSE-VP22-EGFP and 109±12 EGFP-positive cells were counted in those injected with HIV-NSE-EGFP. A total number of 197±18 EGFP-positive cells for HIV-CMV-VP22-EGFP and 76±11 EGFP-positive cells for HIV-CMV-EGFP, respectively, were also counted per slide in the mice brains. We found that the distribution of EGFP-positive cells in the hippocampus injected with the lentiviral vectors with VP22 were significantly higher than those injected with the lentiviral vectors without VP22 (FIG. 8B). We also observed that VP22-EGFP fusion protein was transported via axons between the neurons in the hippocampus. Immunofluorescence assays indicated that most of the transduced CNS cells were neurons. Furthermore, we discovered that the lentiviral vectors have the capacity to infect astrocytes in the CNS in vivo.

### Immunoassay of Transduced Target Cells in CNS

Confocal microscopy and double immunofluorescence detection were used to assess the cell types expressing EGFP in the mouse central nervous system. In all animals, approximately 70% of CNS cells transduced by the lentiviral vectors under the control of CMV promoter were NeuN-immunoreactive neurons; they colocalized with the cells that expressed EGFP. Moreover, more than 90% of the cells transduced by the vector under the control of NSE promoter were neurons in the CNS (FIG. 8); 9.6% of the glial fibrillary acidic protein (GFAP)-immunoreactive astrocytes were transduced by the lentiviral vectors in the CNS. The data indicated that EGFP fluorescence was also found in neurofilament in the CNS.

### Gene Transfer into the CNS in vivo Using a Recombinant Lentivirus Vector

In order to elicit a sufficient therapeutic response in target tissues in vivo, we constructed a recombinant VP22 fusion protein in an HIV-1-based lentiviral vector that delivered its gene product from transduced cells that had been implanted into the CNS. To further investigate gene transfer to cells of the CNS and VP22-EGFP transported between neurons in vivo, we injected the viral vectors directly into the mouse brain. The expressions of reporter genes and fusion protein driven by either a CMV promoter or NSE promoter were also determined. The present in vivo data are consistent with our previous in vitro results that demonstrated VP22 enhanced intercellular trafficking to many neighboring cells. In order to compare the expression profiles of our lentiviral vectors in the CNS, the animals were sacrificed and the brains were collected 3 months after injection, since one of our goals in this study was to determine the long-term expression of our lentiviral vectors. We found that the expression of transgene reached a steady stable expression level at this time point. When the viral vectors were injected directly into the CNS, we found a large number of EGFP positive cells transduced by the lentiviral vector HIV-VP22-EGFP as compared to the injection of HIV-EGFP. In the striatum, injection of HIV-VP22-EGFP resulted in widespread TGFβ transport originating from the injection site, whereas EGFP expression showed limited distribution in the nucleus accumbens when injected with HIV-EGFP. Direct injection of the VP22-EGFP vector into the hippocampus resulted in wide-spread distribution of VP22-EGFP as well. Expression was observed from the site of injection in CA2 to the entire layer of pyramidal cells, as well as in the neighboring oriens layer of the hippocampus. Injection with the vector lacking VP22 revealed that EGFP expression was essentially restricted to the site of injection with very limited diffuse expression in less than one-third of the pyramidal cell layer. Although the titer for HIV-NSE/CMV-VP22-EGFP (mean value: 1.0×10⁶) was lower than that of HIV-NSE/CMV-EGFP (mean value: 3.0×10⁶), the distribution of EGFP in both the striatum and hippocampus injected with the lentiviral vectors including VP22 was significantly higher than those injected with the viral vector without VP22. Thus, the spread of EGFP in these tissues was due to the transporting function of VP22-fusion protein delivered by the lentiviral vectors. This finding was further confirmed by counting the numbers of EGFP-positive cells in the striatum in vivo (FIG. 8A), as well as in the hippocampus (FIG. 8B).

Using confocal microscopy combined with an advanced computer program, we found that the total numbers of EGFP-positive neuronal cells in either the striatum or hippocampus injected with the lentiviral vectors with VP22 were significantly higher than the numbers of transduced neuronal cells when injected with the lentiviral vectors without VP22 (FIG. 8). Immunohistochemistry indicated that most of the transduced cells were neurons. Cell-to-cell transport of VP22-EGFP fusion protein via axons was also observed in vivo. EGFP fluorescence was also found in association with neurofilaments in 9.6% of the astrocytes. The latter finding suggests that the lentiviral vector has the capacity to deliver the transgene product into astrocytes in vivo. Moreover, structure relationships between neurons and astrocytes in the CNS were clearly illustrated by the EGFP fluorescence in vivo. This finding confirmed the suggestions that our lentiviral vector driven by NSE promoter is also an investigative tool for further understanding the structure and function of the cells in specific systems within the CNS. Interestingly, we found that using the neuronal promoter, 30% more EGFP positive cells were observed than the CMV promoter. Our data also demonstrated that up to 90% of the CNS cells transduced by the lentiviral vector controlled by the neuronal promoter are neurons. This finding indicates that the NSE promoter is stronger in driving gene expression than the CMV promoter in vivo, particularly for neurons in the CNS. Therefore, a tissue or cell-specific promoter such as NSE promoter in this vector system should increase the efficiency and potency of gene products or proteins delivered by the lentivirus. This is envisioned as being very helpful for targeting specific tissues without toxicity and immune response.

### EXAMPLE 1

Vector Constructs. Referring to FIG. 1, the double-gene HIV-EGFP/HSA vector is described below. The pUL49ep clone encoding VP22 was provided by J. McLauchlan (Institute of Virology, Glasgow, Scotland; Leslie, J. et al. 1996 Virology 220:60-68). The pLL49ep BamHI fragment was cloned in frame to the EGFP coding region present in pEGFP-N1 (Clontech). A DNA fragment encoding VP22 fused to EGFP was subsequently subcloned into HIV-EGFP/HSA to yield HIV-VP22-EGFP/HSA. Virus was produced in 293T cells by transient transfection as described (Reiser, J. et al. 1996 PNAS USA 93:15266-15271; Mochizuki, H. et al. 1998 J Virol 72:8873-8883). Virus stocks were concentrated by ultracentrifugation.

Referring to FIG. 1, 2, 3, 4, 5, and 6, the following plasmids were obtained through the AIDS Research and Reference Program, Division of AIDS, National Institute of Allergy and Infectious Diseases (NIAID), National Institutes of Health, Bethesda, Md.: pHIVgpt from Kathleen Page and Dan Littman (Page, K. A. et al. 1990 J Virol 64:5270-5276), pNL4-3 from Malcom Martin (Adachi, A. et al. 1986 J Virol 59:284-291), and pNL4-3.HSA.R⁻E⁻ from Nathaniel Landau (He, J. et al. 1995 J Virol 69:6705-6711). All nucleotides are numbered in accordance with Korber et al. (Korber, B. et al. 1998 Human retroviruses and AIDS 1998. A compilation and analysis of nucleic acid and amino acid sequences. Theoretical Biology and Biophysics Group, Los Alamos National Laboratory, Los Alamos, N. Mex.). The two-gene HIV-EGFP-HSAΔE vector is based on the original HIV-EGFPΔE vector (Mochizuki, H. et al. 1998 J Virol 72:8873-8883). The sequences between the BamHI (position 8464) and Ahol (position 8886) sites were replaced with the BamHI/XhoI fragment from pNL4-3.HSA.R⁻E⁻ carrying the HSA reporter gene within the nef coding region (He, J. et al. 1995 J Virol 69:6705-6711). The HIV-EGFP-HSAΔE tat(−) vector contains two consecutive termination codons after amino acid 10 within the 5′ tat exon. It is based on the pTat(−)GV/4GS™ construct (Huang, L. M. et al., 1994 EMBO J. 13:2886-2896) that was provided by K.-T. Jeang (NIAID). The HIV-EGFP-HSAΔE rev(−) vector encodes a truncated version of Rev. It was created by filling up the unique BamHI site present within rev exon 2 using T4 DNA polymerase, leading to a 4-bp insertion. The HIV-EGFP-HSAΔE tat(−) and HIV-EGFP-HSAΔE rev(−) vectors were combined to yield HIV-EGFP-HSAΔE tat(−)/rev(−). The three-gene HIV-EGFP-neo-HSAΔE vector was derived from the original HIV-neoΔE construct (Mochizuki, H. et al. 1998 J Virol 72:8873-8883). An expression cassette consisting of the human cytomegalovirus (CMV) immediate-early (IE) promoter linked to the EGFP coding region was derived from pEGFP-C1 (Clontech) and inserted between the NsiI (position 1247) and EcoRI (position 5743) sites, and the sequences between the BamHI and XhoI sites were replaced with sequences carrying the HSA coding region as described above. The bicistronic HIV-HSA-IRES-EGFPΔE vector was constructed as follows. The gag, pol, vif, and vpr sequences between the SpeI (nucleotide 1506) and EcoRI (nucleotide 5742) sites were deleted from the original HIV-HSA construct harboring HSA sequences driven by the CMV IE promoter (Reiser, J. et al. 1996 PNAS USA 93:15266-15271). A 1.34-kb fragment carrying the encephalomyocarditis virus (ECMV) internal ribosome entry site (IRES) sequence (Morgan, R. A. et al. 1992 Nucleic Acids Res 20:1293-1299) and EGFP gene sequences was derived from pIRES-EGFP (Clontech). The fragment was inserted downstream from the HSA coding region at position 7611. All NL vectors are based on the NL4-3 molecular clone (Adachi, A. et al. 1986 J Virol 59:284-291) with the sequences between the NsiI (position 1246) and BglII (position 7611) sites deleted. A 168-bp simian virus 40 (SV40) origin of replication fragment and a 133-bp fragment harboring HIV-1 polypurine tract sequences (Charneau, P. et al. 1994 J Mol Biol 241:651-662) were placed between these two sites (Reiser, J. 2000 Gene Ther 7:910-913). Various expression cassettes were inserted between the BamHI (nucleotide 8464) and XhoI (nucleotide 8886) sites. NL-EGFP carries an expression cassette consisting of the CMV IE promoter linked to the EGFP coding region. NL-HSA carries a similar expression cassette encoding the mouse HSA cDNA. The CEF hybrid promoter was derived from pCE-490 (SnaBI-BamHI fragment) (Takada, T. et al. 1997 Nat Biotechnol 15:458-461). To construct the NL-HSA-IRES (ECMV)-EGFP and NL-HSA-IRES (ECMV)-EGFP/CEF bicistronic vectors, a fragment carrying the HSA and EGFP genes linked by an ECMV IRES sequence was used as described above. The NL-HSA-IRES (Gtx)-EGFP vector contains an IRES [(Gtx133-141)₁₀(SI)₉β; 208-bp SpeI/NcoI fragment] derived from the 5′ untranslated region of the mRNA encoding the Gtx homeodomain protein (Chappell, S. A. et al. 2000 PNAS USA 97:1536-1541).

Referring to FIG. 7, a polymerase chain reaction (PCR) fragment of a neuron specific enolase (NSE) promoter/EGFP obtained from adeno-associated vector, AAV/NSE-EGFP was introduced into lentiviral vector, HIV/CMV (cytomegalovirus promoter)-EGFP between the AseI and the BsrGI sites. Virus production and transduction of cells were described previously (Reiser, J. et al. 1996 PNAS USA 93:15266-15271; Mochizuki, H. et al. 1998 J Virol 72:8873-8883). In brief, pseudotyped virus was generated by transfection of plasmid DNA into 293T cells or COS-7 cells by calcium-phosphate precipitation. Virus stocks were concentrated by ultracentrifugation. The lentivirus stocks were generated with the following titers (pfi/ml): 1.0×10⁶ for HIV-NSE-VP22-EGFP, 3.1×10⁶ for HIV-NSE-EGFP, and 0.9×10⁶ for HIV-CMV-VP22-EGFP, 2.9×10⁶ for HIV-CMV-EGFP.

Virus Production. Vector particles pseudotyped with the vesicular stomatitis virus G glycoprotein (VSV-G) were produced using a three-plasmid expression system by transient transfection of human 293T cells with a defective packaging construct (Mochizuki, H. et al. 1998 J Virol 72:8873-8883), a plasmid with the VSV-G coding region driven by the HIV LTR (Reiser, J. et al. 1996 PNAS USA 93:15266-15271) and a HIV-1 based vector construct. Five micrograms of each of the three plasmid DNAs were cotransfected into subconfluent 293T cells using the calcium phosphate precipitation method. Cells were seeded into six-well plates 24 to 30 hrs prior to transfection. Chloroquine (25 μM final concentration) was added to the cells immediately before transfection, and the medium was replaced with 2 ml (per well) of fresh DMEM supplemented with 10% FBS 12 to 14 h later. The virus was harvested 60 to 65 h later, filtered through a Millipore Millex-HA 0.451μ filter unit, aliquoted, and frozen at −80° C. p24 assays were performed using a commercial kit (Cellular Products Inc.). The generation of replication-competent virus was tested by serially passaging transduced H9 cells over a period of 4 weeks followed by measurement of p24 levels (Mochizuki, H. et al. 1998 J Virol 72:8873-8883).

Animals. Adult mice (57/BL16, 25 g), obtained from Taconic Farms, were maintained in a BSL2/3 animal facility in a temperature- and light-controlled room, with food and water available ad libitum. The mice were anesthetized with Avertin solution (Aldrich) i.p. (0.15 ml/10 g body weight) before injection. They were placed in a small-animal stereotactic apparatus fitted to a mouse adaptor with the skull horizontal between lambda and bregma. Following the surgery and injection, the animal's scalp was closed and sterilized before return to the recovery cage. The animal experiment was approved by the Animal Care and Use Committee at the National Institutes of Health.

Cell Culture and Infection. Human embryonic kidney 293T cells (DuBridge, R. B. et al. 1987 Mol Cell Biol 7:379-387) were provided by Warren Pear (Rockerfeller University). Human osteosarcoma (HOS) cells, primary human skin fibroblasts (HSFs), and human endothelial, brain choroid plexus, HeLa, and COS-7 cell lines were obtained from the American Type Culture Collection. The human H9 cell line was obtained from Dr. Robert Gallo (Popovic, M. et al. 1984 Science 224:497-500) through the AIDS Research and Reference Program, Division of AIDS, National Institute of Allergy and Infectious Diseases, National Institutes of Health. The COS-7, HOS, and HeLa cells were grown in DMEM (GIBCO) containing 10% heat-inactivated FBS. Human endothelial cells were grown in F12 K-medium with 2 mM L-glutamine containing 1.5 g/liter sodium bicarbonate, 100 μg/ml heparin, 30 μg/ml endothelial cell growth supplement (ICN), and 10% FBS. The brain choroid plexus cells were grown in Eagle's minimum essential medium with 0.1 mM nonessential amino acids, 90% Earle's balanced salt solution, and 10% FBS. H9 cells were grown in 80% RPMI 1640 medium (GIBCO) containing 10% FBS, 2×L-glutamine, 0.05 mg/ml gentamicin and 1×Penstrep (GIBCO). Cells were infected in DMEM/FBS containing 4 μg/ml Polybrene for 4-16 h.

Immunofluorescence Analysis and Flow Cytometry. Approximately 2×10% COS-7 or HOS cells per well were plated into six-well plates and infected with 0.25 ml of virus. Infected COS-7 cells were trypsinized 24 h after infection and coplated with human endothelial cells, brain choroid plexus cells, and HeLa cells at a ratio of 1:10, then allowed to grow for 3 d. Cells were grown on 12-mm, round coverslips coated with poly-L-lysine (Becton Dickinson) in 12-well culture dishes in 2.2 ml of medium. Cells were fixed with 4% paraformaldehyde in 1×Hanks' balanced salt solution (HSS, GIBCO) containing 2% FBS for 10 min at room temperature. The samples were washed three times with 1×HSS, and blocked with 10% goat serum in 1×HSS for 20 min at room temperature. Monoclonal mouse simian virus 40 T-antigen antibody (Calbiochem) was added at a dilution of 1:100 and the cells were incubated for 60 min at room temperature. The samples were then washed three times with 1×HSS, and incubated with a secondary anti-mouse antibody conjugated with tetramethylrhodamine isothiocyanate (TRITC; Sigma) for 30 min at room temperature. The coverslips were carefully removed after washing three times with 1×HSS, and then mounted on slides for microscopic observation. Statistical evaluation was performed by using a Student's unpaired t test (Statwork, Microsoft). Mean values for the numbers of cells with positive fluorescent staining were determined by averaging values from three experiments.

For immunofluorescence staining of H9 cells, a phycoerythrin-labeled monoclonal anti-IL-2 receptor (IL-2R) antibody (PharMingen, Calif.) was used. The dish containing transduced COS-7 cells was first washed three times with culture medium. The suspension of H9 cells was then directly transferred onto a monolayer of transduced COS-7 cells. The suspension of H9 cells was collected 3 d after coculturing, and washed three times with 1×HSS solution containing 5% FBS. The resuspended cells were then transferred to a 50-mm tube in which antibody staining (1:100 dilution) was carried out for 30 min on ice. The cells were washed three times with PBS/FBS buffer, and 0.1-0.2 ml of diluted cells (5×10⁵) was placed in a Cytospin block. The blocks were centrifuged at 800 rpm for 5 min. After removal from the blocks and fixing in ethanol-glacial acetic acid for 15 min at −20° C., the slides were analyzed by Zeiss Axiophot fluorescence microscopy equipped with a Hamamastu charge-coupled device camera.

For fluorescence-activated cell sorter (FACS) analysis, cells were detached from the plate by using PBS containing 2 mM EDTA 3 d after infection, and then incubated with a phycoerythrin-labeled anti-HSA monoclonal antibody (1:40 dilution) for 30 min on ice. The cells were collected after centrifugation and resuspended in PBS for subsequent FACS analysis.

Implantation of Transduced Cells Into Mouse Brain Ventricles. The animals were divided into two groups (5 animals per group). The first group was implanted with transduced cells previously infected by using the HIV-EGFP/HSA vector. The second group was implanted with cells previously infected by using the HIV-VP22-EGFP/HSA vector. Transduced H9 cells were washed with PBS in 1×HSS containing 0.2% trypsin and subsequently washed two times with PBS in 1×HSS. The cells were then concentrated by centrifugation for implantation. The animal was anesthetized and the head was fastened in the stereotactic apparatus. Injections of transduced cells into the lateral ventricles of the brain were performed at the following coordinates: 0.38 mm to bregma, 0.65 mm to the midline, and 3.0 mm depth. Twenty microliters of the transduced cells (10⁶-10⁷ cells per ml) was loaded into an internal cannula needle (23 gauge) with cannula tubing connected to a Hamilton syringe mounted on a microinjection pump (Harvard Apparatus). The cells were delivered into the ventricle of the brain at a rate of 1.0 μl/min.

Vector Injection into the Mouse Brain. Mice were divided into four groups (5 animals per group). The first and the second groups were injected with either the HIV-NSE-EGFP or HIV-CMV-EGFP lentiviral vectors. The third and the fourth groups received either the HIV-NSE VP22-EGFP or HIV-CMV-VP22-EGFP lentiviral vectors, respectively. The procedure for surgery was as described above using the following coordinates: for injection into the striatum: 1.70 mm anterior to the bregma, 1.1 mm to the right of the midline, and 4.1 mm depth; for injection into hippocampus: 2.3 mm anterior to the bregma, 1.0 mm to the right of the midline, and 2.0 mm depth. Three microliters of concentrated viral vectors were loaded into an internal cannula needle (C315×33) with cannula tubing connected to a Hamilton syringe mounted on a microinjection pump (Harvard Apparatus, Dover, Mass.). The viral vector solutions were delivered at a rate of 0.5 μl/min.

Brain Immunofluorescence Assay. Animals were sacrificed by decapitation 3 months after injection and whole brains were carefully removed. The brains were immediately fixed with 4% paraformaldehyde/1% glutaraldehyde for 24 h at 4° C., then washed with phosphate-buffered saline (PBS) in 1×Hank's balanced salt solution (HSS) containing 4% sucrose for 2 d at 4° C. The tissues were embedded in O.C.T. (optimum cutting temperature) medium (Tissue-Tek, Miles Inc., Indianapolis, USA) and frozen in a methanol/dry ice bath. The frozen tissues were sectioned to a thickness of 15 μm per coronal section by using a cryostat (Bright Instrument, Huntingdon, UK) at −18° C. For immunocytochemical detection of implanted cells, the brain sections were washed three times with PBT buffer (PBS in 1×HSS, 0.1% bovine serum albumen (BSA) and 0.2% Tween 20), then blocked with 10% goat serum for 15 min. After washing three times with PBT buffer, slides were incubated in the phycoerythrin-labeled monoclonal anti-IL-2R antibody (1:500; PharMingen, Calif.) for 45 min at room temperature. The slides were washed with PBT buffer and analyzed by using a Zeiss 510 confocal microscope.

For immunocytochemical detection of neurons, astrocytes and neurofilaments, the brain sections were washed three times with PBT buffer (PBS in 1×HSS, 0.1% BSA and 0.2% Tween 20), then blocked with 10% goat serum for 15 min. After washing three times with PBT buffer, slides were incubated with primary murine antibodies against the NeuN (neuron-specific nuclear protein, 1:200; Chemicon, Temecula, Calif.), glial fibrillary acidic protein (GFAP; 1:400; Chemicon), and neurofilament (NF; 1:200; Chemicon) at 4° C. overnight. The anti-mouse tetramethylrhodamine isothiocyanate (TRITC)-conjugated secondary antibodies (SIGMA, St. Luis, Mo.) were then added onto slides for 30 min at room temperature.

Having now fully described the invention, it will be understood to those of ordinary skill in the art that the same can be performed with a wide and equivalent range of conditions, formulations, and other parameters without affecting the scope of the invention or any embodiment thereof. All patents and publications cited herein are fully incorporated by reference hereby in their entirety.

What is claimed is:

1. A pharmaceutical composition comprising, in combination with a pharmaceutically acceptable excipient, a recombinant lentivirus comprising:
(a) a nucleic acid sequence containing a lentiviral packaging signal flanked by lentiviral cis-acting nucleic acid sequences necessary for reverse transcription and integration;
(b) a heterologous nucleic acid sequence operably linked to a regulatory nucleic acid sequence; and
(c) a nucleic acid sequence encoding an intercellular trafficking signal;
wherein the nucleic acid sequence encoding the intercellular trafficking signal is fused in-frame with the heterologous nucleic acid sequence; and
wherein the lentivirus does not contain a complete gag, pol, or env gene.

2. A method of making a pharmaceutical composition comprising producing a recombinant lentivirus and combining it with a pharmaceutically acceptable excipient, wherein the producing step comprises:
(a) transfecting a suitable packaging host cell with the following vectors:
(i) a first vector providing a nucleic acid encoding a lentiviral gag and a lentiviral pol, where the gag and pol nucleic acid sequences are operably linked to a heterologous regulatory nucleic acid sequence and where the first vector is defective for nucleic acid sequence encoding functional env protein and devoid of lentiviral sequences both upstream and downstream from a splice donor site to a gag initiation site of a lentiviral genome;
(ii) a second vector providing a nucleic acid encoding a non-lentiviral env protein; and
(iii) a third vector providing a nucleic acid sequence containing a lentiviral packaging signal flanked by lentiviral cis-acting nucleic acid sequences for reverse transcription and integration, a heterologous nucleic acid sequence operably linked to a regulatory nucleic acid sequence, and a nucleic acid sequence encoding an intercellular trafficking signal, wherein the nucleic acid sequence encoding the intercellular trafficking signal is fused in-frame with the heterologous nucleic acid sequence, and wherein the third vector does not contain a complete gag, pol, or env gene; and
(b) recovering the recombinant lentivirus.

3. The pharmaceutical composition of claim 1 wherein the recombinant lentivirus further comprises functional tat and rev coding regions and wherein the heterologous nucleic acid sequence operably linked to the regulatory nucleic acid sequence forms an expression cassette that is placed 5′ to the Rev-responsive element (RRE).

4. The pharmaceutical composition of claim 1 wherein the recombinant lentivirus further lacks tat and rev coding regions and wherein the heterologous nucleic acid sequence operably linked to the regulatory nucleic acid sequence forms an expression cassette that is placed 3′ to the Rev-responsive element (RRE).

5. The pharmaceutical composition of claim 1 wherein the recombinant lentivirus further comprises a second heterologous nucleic acid sequence operably linked to a regulatory nucleic acid sequence to form a double gene vector.

6. The pharmaceutical composition of claim 5 wherein the recombinant lentivirus further comprises a third heterologous nucleic acid sequence operably linked to a regulatory nucleic acid sequence to form a triple gene vector.

7. The pharmaceutical composition of claim 1 wherein the recombinant lentivirus further comprises a second heterologous nucleic acid sequence operably linked to the first heterologous nucleic acid sequence by an internal ribosome entry site (IRES) sequence to form a bicistronic expression cassette.

8. The pharmaceutical composition of claim 7 wherein the recombinant lentivirus further comprises functional tat and rev coding regions and wherein the bicistronic expression cassette is placed 5′ to the Rev-responsive element (RRE).

9. The pharmaceutical composition of claim 7 wherein the recombinant lentivirus further lacks tat and rev coding regions and wherein the bicistronic expression cassette is placed 3′ to the Rev-responsive element (RRE).

10. The pharmaceutical composition of claim 1 wherein the recombinant lentivirus further comprises a second heterologous nucleic acid sequence operably linked to a regulatory nucleic acid sequence, further comprises functional tat and rev coding regions and wherein the first heterologous nucleic acid sequence operably linked to the regulatory nucleic acid sequence forms an expression cassette that is placed 5′ to the Rev-responsive element (RRE) and within the env coding region, and wherein the second heterologous nucleic acid sequence is placed 3′ to the Rev-responsive element (RRE) and within the nef coding region.

11. The pharmaceutical composition of any of claims 1-10 (excluding 2) wherein the intercellular trafficking signal is VP22 or a fragment or homologue thereof that retains a VP22 intercellular transport function.

12. The pharmaceutical composition of any of claims 1-10 (excluding 2) wherein the intercellular trafficking signal is VP22.

13. The pharmaceutical composition of any of claims 1-10 (excluding 2) wherein the recombinant lentivirus is HIV-1.

14. The pharmaceutical composition of any of claims 1-10 (excluding 2) wherein the regulatory nucleic acid sequence comprises an enhancer selected from Table 1.

15. The pharmaceutical composition of any claims 1-10 (excluding 2) wherein the regulatory nucleic acid sequence comprises a promoter element selected from Table 2.

16. The pharmaceutical composition of any of claims 1-10 (excluding 2) wherein the heterologous nucleic acid sequence is a cloned structural gene selected from Table 3.

17. The pharmaceutical composition of any of claims 1-10 (excluding 2) wherein the heterologous nucleic acid sequence encodes a heterologous protein selected from Table 4.

18. The pharmaceutical composition of any of claims 1-10 (excluding 2) wherein the heterologous nucleic acid sequence encodes a heterologous protein selected from the group consisting of glucocerebrosidase useful in the treatment of Gaucher Disease, hexosamimidase useful in the treatment of Tay-Sachs Disease, galactocerebrosidase useful in the treatment of Krabbe's Disease, sphingomyelinase useful in the treatment of Niemann Pick Disease, beta-galactosidase useful in the treatment of Gangliosidosis Disease, duronidase useful in the treatment of Hurler Disease, and duronate sulphatase useful in the treatment of Hunter Disease.

19. A method for introduction and expression of a heterologous nucleic acid sequence in a non-dividing cell in vivo comprising infecting the non-dividing cell with the pharmaceutical composition of any of claims 1-10 (excluding 2) and expressing the heterologous nucleic acid sequence in the non-dividing cell in vivo.

20. A method of screening for drugs that downregulate the nef gene comprising infecting a cell with the pharmaceutical composition of claim 10 and measuring downregulation of the second heterologous nucleic acid sequence by the first heterologous nucleic acid sequence.
